(19) United States
(12) Patent Application Publication
(10) Pub. No.: US 2014/0024624 A1
(43) Pub. Date: Jan. 23, 2014

(54) Method of Ameliorating Clotting Pathologies and Related Materials and Methods

(71) Applicant: THE OHIO STATE UNIVERSITY, Columbus, OH (US)

(72) Inventors: Chandan K. Sen, Upper Arlington, OH (US); Sashwati Roy, Upper Arlington, OH (US); Savita Khanna, Columbus, OH (US); Cameron Rink, Lewis Center, OH (US)

(73) Assignee: THE OHIO STATE UNIVERSITY, Columbus, OH (US)

(21) Appl. No.: 13/837,021

(22) Filed: Mar. 15, 2013

Related U.S. Application Data
(60) Provisional application No. 61/673,500, filed on Jul. 19, 2012.

(51) Int. Cl. A61K 31/353; A23L 1/30; A61K 31/445; A61K 45/06; A61K 31/616

(52) U.S. Cl. CPC A61K 31/353; A61K 45/06; A61K 31/616; A61K 31/445; A23L 1/30
USPC 514/165; 514/456; 514/327; 514/262.1

(57) ABSTRACT

Methods for reducing blood clotting in a subject are provided, including methods to reduce the risk of and/or treat blot clotting pathologies. Also provided are compositions useful to reduce the risk or treat blood clotting pathologies.

### CROSS REFERENCE TO RELATED APPLICATIONS

This application claims the benefit of U.S. Provisional Patent Application No. 61/673,500 filed Jul. 19, 2012, the entire disclosure of which is expressly incorporated herein by reference.

### BACKGROUND OF THE INVENTION

Natural vitamin E is a fat-soluble vitamin that exists in eight different forms. Each form has its own biological activity, which is the measure of potency or functional use in the body. Vitamin E is a dietary antioxidant that assists in maintaining cell integrity. It is obtained from sunflower, safflower, canola, and olive oils; also from many grains, nuts, fruits as well as fatty parts of meats.

Natural vitamin E comprises two general types of compounds: tocopherols (80%) and tocotrienols (20%). Both tocopherols and tocotrienols possess a chromanol ring. Tocopherols are characterized by a saturated side chain, whereas tocotrienols possess an isoprenoid side chain with double bonds at C-3, -7 and -11.

Clinical trials testing the effects of natural vitamin E in a wide range of major health disorders have come to the general conclusion that natural vitamin E either is not helpful or could be harmful under certain conditions.

Palm oil represents a major source of natural tocotrienols. Tocotrienols possess powerful neuroprotective, antioxidant, anti-cancer and cholesterol lowering properties that are biologically unique from the properties of tocopherols and work independent of antioxidant function. Micromolar amounts of tocotrienols suppress the activity of HMG-CoA reductase, the hepatic enzyme responsible for cholesterol synthesis. Tocotrienols are thought to have more potent antioxidant properties than tocopherols. The unsaturated side chain of tocotrienols allows for more efficient penetration into tissues that have saturated fatty layers such as the brain and liver. Comparative examination of the antioxidant properties of tocopherols and tocotrienols revealed that tocotrienols are advantageous because of their better distribution in the fatty layers of the cell membrane. Recently, an antiangiogenic function of tocotrienol has been reported.

Like tocopherols, tocotrienols have been identified to possess distinct functions that may benefit human health, yet tocotrienols account for a very small fraction of overall vitamin E research.

Approximately 795,000 strokes and >200,000 transient ischemic attacks (TIAs) occur in the US each year with 7 million stroke and TIA survivors. For stroke survivors, there is an 11% risk of recurrent stroke at 1 year and 26% risk at 5 years. The risk of stroke within 90 days of a TIA is between 9-11%.

Some recurrent cardiovascular events including stroke seen in patients with cardiovascular disease on aspirin and clopidogrel may relate to the failure of these therapies to inhibit platelet aggregation in vitro. The incidence of aspirin and clopidogrel resistance is estimated to be 20%.

In the U.S., it is estimated that ˜30 million people (9.5% of the population) take aspirin, and ˜48 million (15.3%) take clopidogrel daily for cardiovascular protection. Given that as high as 20% of the population are resistant to these therapies, millions of Americans may benefit from an alternate therapy.

Aspirin decreases the relative risk of recurrent stroke, myocardial infarction, and vascular death by 13%, and non-fatal ischemic stroke by 19% in patients who have had a TIA or stroke. Anti-platelet agents such as clopidogrel and extended release dipyridamole plus aspirin are modestly more effective than aspirin alone in preventing stroke or combined cardiovascular endpoints; however, these medications are not as effective as dose-adjusted warfarin in patients with stroke due to atrial fibrillation where relative stroke risk is decreased by 62%. High dose atorvastatin decreases cholesterol levels in patients with stroke and decreases the risk of recurrent stroke but only by about 2% over 5 years. Also chronic treatment paradigms for aspirin, clopidogrel, and statins are not without detrimental side effects that include gastrointestinal ulcers, neutropenia, myalgias, and elevated liver function tests.

### SUMMARY OF THE INVENTION

The present invention provides methods of reducing blood clotting in a subject in need thereof, comprising administering a pharmaceutically-effective formulation of at least one isoform of tocotrienol and reducing blood clotting in a subject.

Also provided are such methods, wherein the formulation comprises a dose of tocotrienol selected from the group consisting of: approximately 10 mg to approximately 1000 mg per day; approximately 50 mg to approximately 500 mg per day; approximately 100 mg to approximately 500 mg per day; approximately 200 mg to approximately 500 mg per day; approximately 300 mg to approximately 500 mg per day; up to approximately 400 mg per day.

Also provided are such methods, wherein the formulation comprises a dose of tocotrienol selected from the group consisting of: approximately 200 mg to approximately 1000 mg in one weekly dose; approximately 300 mg to approximately 1000 mg in one weekly dose; approximately 400 mg to approximately 500 mg in one weekly dose.

Also provided are such methods, wherein the formulation is administered as a dose regimen selected from the group consisting of: approximately every other day; approximately every third day; approximately every fourth day; approximately every fifth day; approximately every sixth day; approximately every seventh day; approximately every other week; approximately once a month; approximately twice a month; approximately three times a month; approximately four times a month; approximately five times a month; approximately six times a month; approximately seven times a month; and approximately eight times a month.

Also provided are such methods, wherein the formulation is an orally-delivered supplement.

Also provided are such methods, wherein the oral supplement is delivered by one or more of: a capsule, a tablet pill, a colloid, a piece of chewing gum, a gel, a drink, a food additive, a thin film dissolving strip, an emulsified food spread, an emulsion, a syrup, a meat food, a dairy food, and an egg.

Also provided are such methods, wherein the subject is selected from a population at elevated risk for a blood clotting pathology selected from the group consisting of: transient ischemic attack (TIA); stroke; embolism; and cardiac dysfunction.

Also provided are such methods, wherein the subject is selected from the group consisting of: a person who has suffered a previous blood clotting pathology; a person who has suffered a stroke; a person who has suffered a cardiac event; a person who has suffered from transient ischemic attack; a person who has suffered an embolism; a person who has suffered a thrombosis; a person with a genetic predisposition for pathologic blood clotting; a person with biomarkers for pathologic blood clotting; and a person who takes anticoagulants.

Also provided are such methods, wherein the subject is selected from the group consisting of: astronaut, pilot, professional racecar driver, deep-sea diver, mountain climber, pre-surgery patient, sickle-cell anemia patient, sleep apnea patient, drug rehabilitation patient, elderly person, elderly animal, or companion animal.

The present invention also provides pharmaceutical composition for the treatment of blood clotting disorders comprising: one or more antiplatelet-aggregation drugs and one or more tocotrienol compounds.

Also provided are such compositions, wherein the thrombolytic drug is selected from the group consisting of: aspirin; clopidogrel; and dipyridamole.

The present invention also provides methods of reducing reducing risk of a second stroke in a stroke patient in need thereof, comprising administering a pharmaceutically-effective formulation of at least one isoform of a tocotrienol and reducing risk of a second stroke in a stroke patient in need thereof.

Also provided are such methods, wherein the stroke patient is resistant to aspirin or clopidogrel.

Also provided are such methods, which comprise administering up to 400 mg of at least one tocotrienol for at least 30 days, and a pharmaceutically-effective dose of at least one tocotrienol weekly thereafter.

The present invention also provides methods of reducing risk of a second stroke in a stroke patient in need thereof, comprising administering a pharmaceutically-effective formulation of at least one isoform of a tocotrienol and reducing risk of a second stroke in a stroke patient in need thereof.

Also provided are such methods, which further comprise administering to the patient a pharmaceutically-effective amount of aspirin or clopidogrel.

Also provided are such methods, which comprise administering up to 400 mg of at least one tocotrienol for at least 30 days, and a pharmaceutically-effective dose of at least one tocotrienol weekly thereafter.

The present invention also provides method of inhibiting arachidonic acid-mediated/12-LOX related platelet aggregation in a subject in need thereof, comprising administering a pharmaceutically-effective amount of mixed tocotrienols at weekly.

The present invention also provides methods for ameliorating the risk or symptoms of pathologic platelet aggregation in a subject in need thereof and for which aspirin is contraindicated, comprising administering a pharmaceutically-effective amount of mixed tocotrienols to the subject.

Also provided are such methods, wherein aspirin is contraindicated for a reason selected from the group consisting of: alcoholism; liver pathology; clotting disorder; allergy; salicylate intolerance; asthma; bronchospasms; peptic or intestinal ulcers; diabetes; gastritis; hemophilia; glucose-6-phosphate dehydrogenase deficiency; dengue fever; children or adolescents; kidney disease; hyperuricemia; and gout.

The present invention also provides methods for ameliorating the risk or symptoms of pathologic platelet aggregation in a subject in need thereof and for which clopidogrel is contraindicated, comprising administering a pharmaceutically-effective amount of mixed tocotrienols to the subject.

Also provided are such methods, wherein aspirin is contraindicated for a reason selected from the group consisting of: alcoholism; liver pathology; allergy; poor metabolizer due to cytochrome p450 CYP2C19 variant; stomach or intestinal ulcer; and kidney disorder.

The present invention also provides methods for reducing the risk of a heart attack in a subject in need thereof, administering a pharmaceutically-effective amount of mixed tocotrienols at weekly.

The present invention also provides methods for reducing the risk of a heart attack in a subject in need thereof, administering a pharmaceutically-effective amount of mixed tocotrienols at weekly.

### BRIEF DESCRIPTION OF THE DRAWINGS

The patent or application file may contain at least one drawing executed in color and/or one or more photographs. Copies of this patent or patent application publication with color drawing(s) will be provided by the Office upon request and payment of the necessary fee.

FIGS. 1A-1B. Antiplatelet properties of tocotrienol vitamin E in human blood. Healthy subjects were supplemented with tocotrienol vitamin E (400 mg/day) or vehicle placebo (control) for 4 weeks. Platelet function was assessed by optical platelet aggregometry with the following agonists: arachidonic acid (AA, 0.5 mM), ADP (10 μM), and collagen (2 μg/ml). FIG. 1A. Representative platelet response curves from control and TCT supplemented subject. FIG. 1B. TCT completely prevented AA-mediated platelet aggregation as compared to control. TCT significantly reduced the platelet effect of ADP and collagen as compared to control (42% and 37% respectively).

FIGS. 2A-2D. Natural vitamin E tocotrienol protects against stroke-induced brain injury. (FIG. 2A and FIG. 2B) Effect of 10 week oral supplementation of TCT or vehicle placebo (PBO) on cerebral cortex a-tocopherol and a-tocotrienol concentration. No TCT was detected in brain of PBO supplemented canines TCT supplementation significantly increased α-tocotrienol concentration in cerebral cortex. (FIG. 2C) Stroke-induced infarct volume in response to stroke. DTI=diffusion tensor image taken at 1 h, FLAIR=fluid attenuated inversion recovery taken at 24 h. (FIG. 2D) Representative coronal MR images of canine brain at 1 h and 24 h after stroke. *p<0.05.

FIG. 3. Human whole blood TCT concentration following daily 400 mg oral supplementation. Blood TCT levels determined by HPLC as reporterd¹⁰. Data represent individual values (males n=6, females n=10) and mean±SD at baseline (0 weeks), 6 weeks and 12 weeks. Across time points, levels without a common letter differ, p<0.05.

FIGS. 4A-4C. Aspirin mimetic anti-platelet effects of natural vitamin E TCT. Healthy subjects (n=3) underwent platelet function testing at baseline and following 2 mos of daily oral TCT (400 mg) or placebo (PBO, vehicle) supplementation. (FIG. 4A) Percent aggregation using optical aggregometry with arachidonic acid agonist (AA, 0.5 mM). AA is the same agonist used to test platelet function in response to aspirin. *p<0.05 PBO vs TCT within time point. (FIG. 4B and FIG. 4C) Representative tracings for PBO and TCT subjects following 2 mos supplementation.

FIG. 5. Oral TCT lowers triglyceride, and raises HDL cholesterol in healthy subjects. Blood cholesterol was tested in healthy subjects (n=3) at baseline and following 3 mos of daily oral TCT (400 mg) supplementation. Average total cholesterol for subjects at baseline was 204.0±11.5. A modest (−5.1%±4.5%), but non-significant decrease in total cholesterol was observed after 3 months of supplementation. Triglyceride levels decreased significantly by 32.8%±10.5%, and HDL cholesterol increased significantly by 10.8%±1.4% at 3 months post-supplementation compared to baseline. *p<0.05, paired Student's test.

FIG. 6. Platelet coagulation, day 62 of supplementation.

FIG. 7. Platelet coagulation, day 2 post-supplementation.

FIG. 8. Platelet coagulation, day 5 post-supplementation.

FIG. 9. Platelet coagulation, day 10 post-supplementation.

FIG. 10. Platelet coagulation, day 20 post-supplementation.

### DETAILED DESCRIPTION OF THE INVENTION

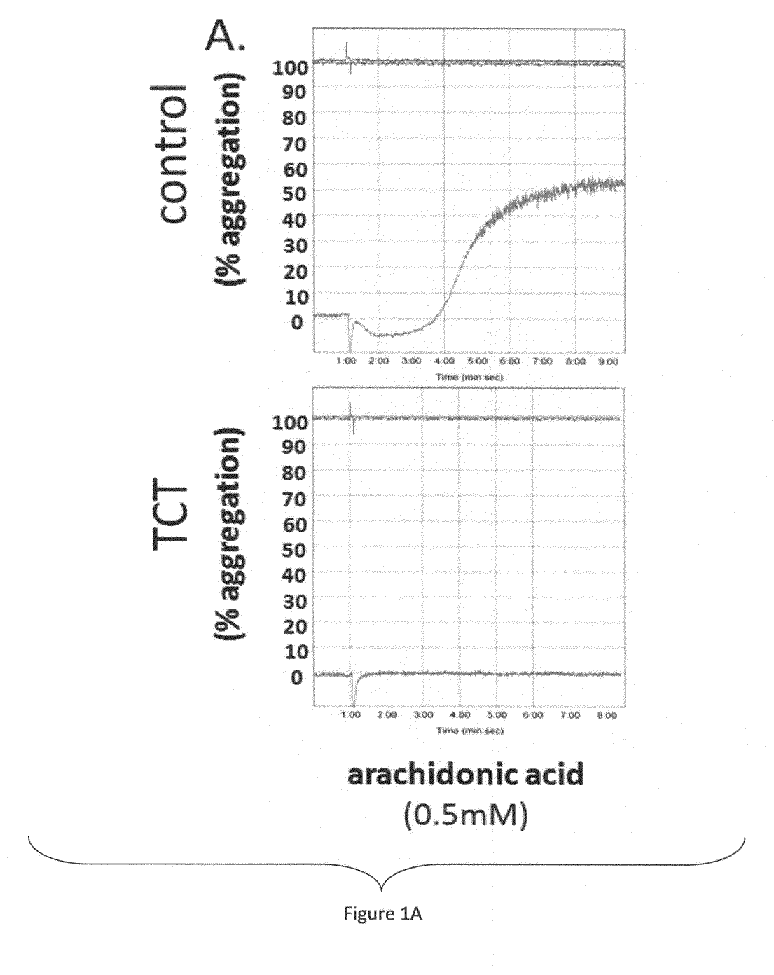

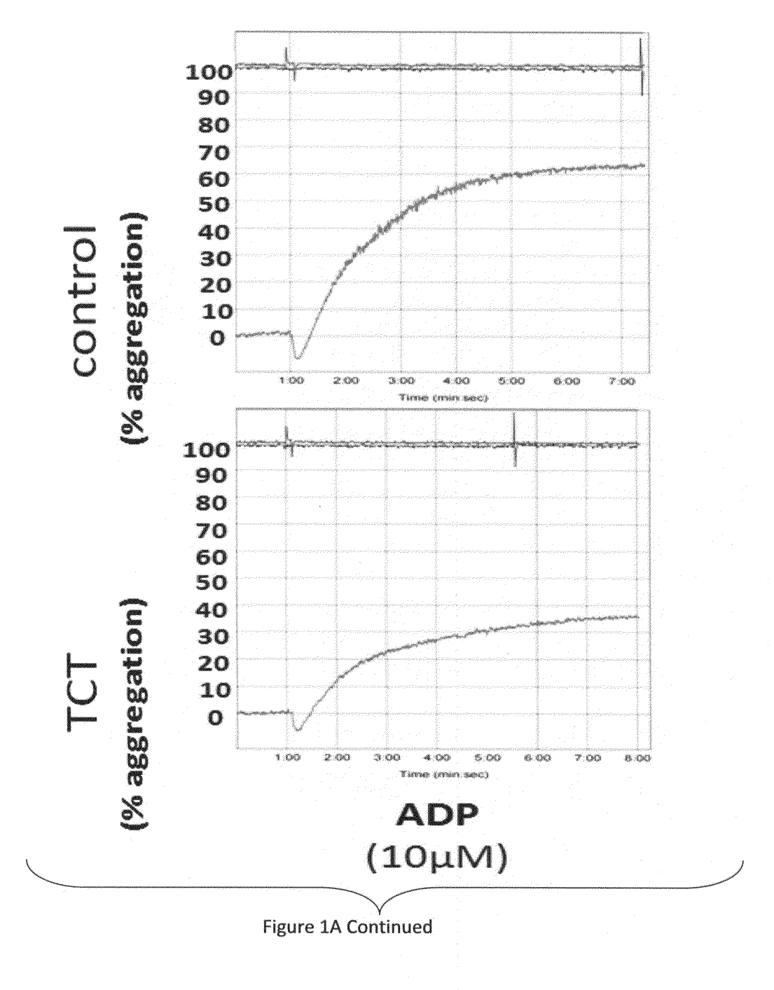

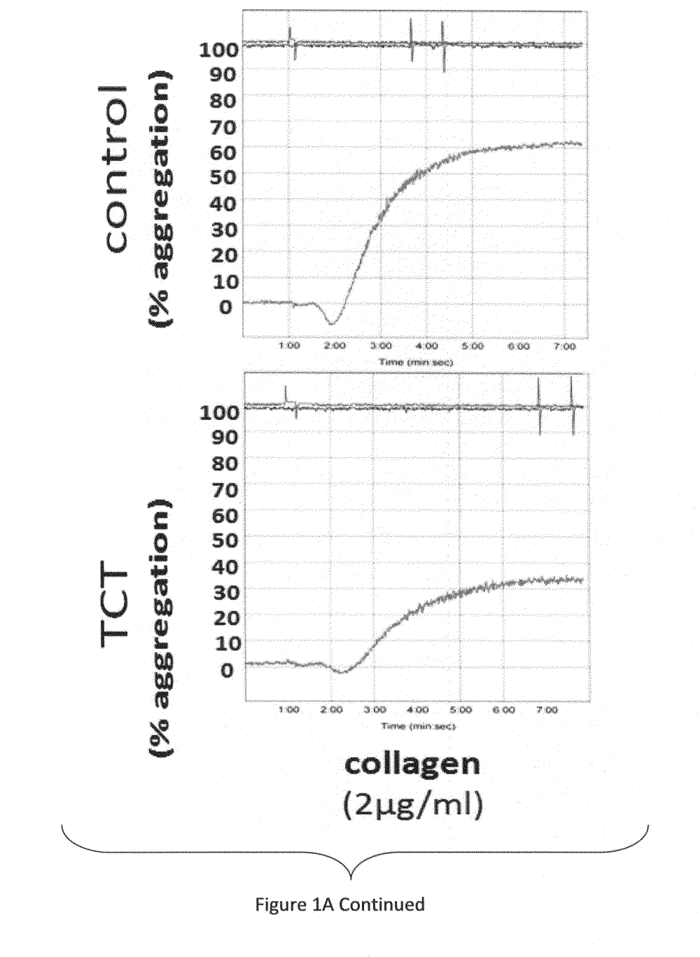

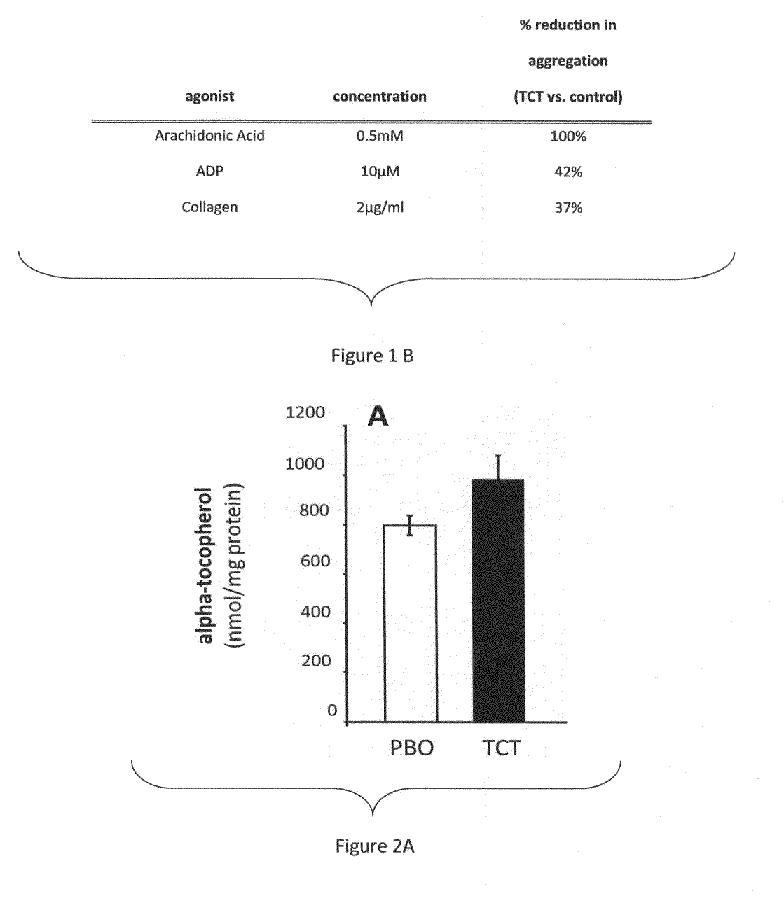

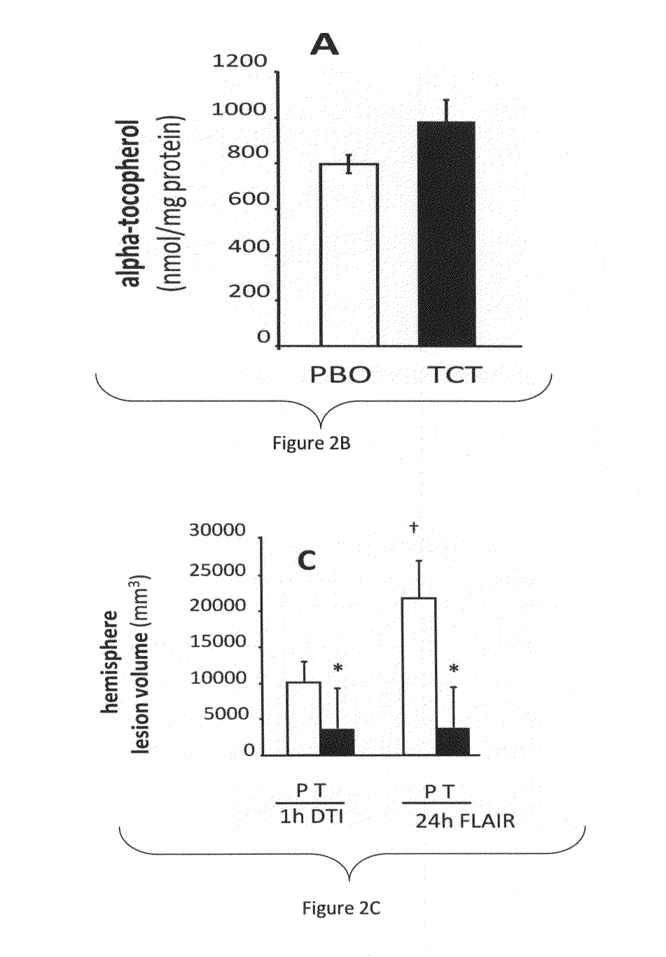

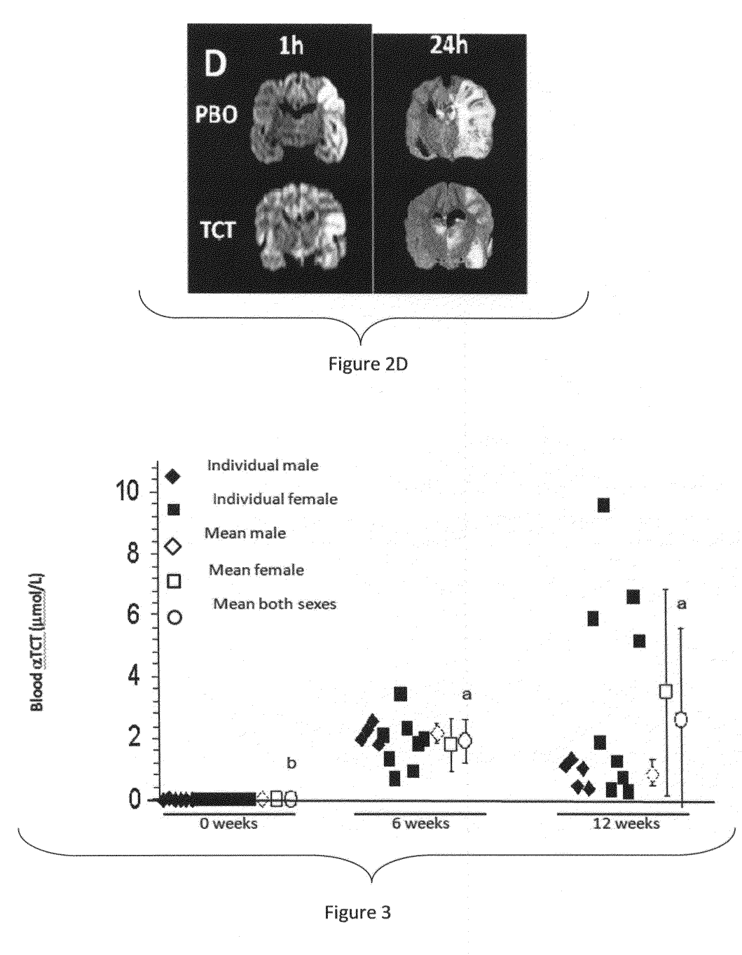

The present invention identifies specific mechanisms that define aspirin-like antiplatelet properties for natural vitamin E isoform alpha-tocotrienol (TCT). When orally supplemented in humans, TCT completely inhibits arachidonic acid-mediated platelet aggregation. TCT also inhibits platelet aggregation induced by other agonists, including ADP and collagen (FIG. 1A).

Aspirin is a drug with known side effects, including hepatotoxicity and ulcerative colitis. Furthermore, aspirin is contraindicated under a number of conditions (including alcoholism or allergies to ibuprofen or naproxen). In contrast, TCT is a natural isoform contained in foods and cooking oils (i.e. palm oil), and has been FDA approved (Generally Recognized As Safe, GRAS certified) for human consumption as a dietary supplement.

Up to 25% of the population do not respond to the antiplatelet effects of aspirin. Aspirin works by acetylation of the active site of cyclooxygenase enzymes (COX-1 and COX-2) thereby inhibiting COX-mediated metabolism of arachidonic acid. TCT targets another enzyme that metabolizes arachidonic acid: 12-Lipoxygenase (12-LOX). This invention is the first to describe an antiplatelet effect for TCT through the 12-LOX pathway. Since TCT works against arachidonic acid mediated platelet aggregation by an alternative mechanism, it benefits aspirin nonresponders.

The practical and commercial applications of the present invention include it being used as a natural antiplatelet alternative to aspirin with no reported side effects.

It can also be used as an alternative with an aspirin-like antiplatelet effect for aspirin non-responders and for patients in which aspirin use is contraindicated (i.e., alcoholism and allergy).

Terms

It is to be understood that both the foregoing general description and the following detailed description are exemplary and explanatory only and are not intended to limit the scope of the current teachings. In this application, the use of the singular includes the plural unless specifically stated otherwise.

The use of the word “a” or “an” when used in conjunction with the term “comprising” in the claims and/or the specification may mean “one,” but it is also consistent with the meaning of “one or more,” “at least one,” and “one or more than one.”

Also, the use of “comprise”, “contain”, and “include”, or modifications of those root words, for example but not limited to, “comprises”, “contained”, and “including”, are not intended to be limiting. The term “and/or” means that the terms before and after can be taken together or separately. For illustration purposes, but not as a limitation, “X and/or Y” can mean “X” or “Y” or “X and Y”.

The term “combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB.

Patient: As used herein, the term “patient” includes human and non-human animals. The preferred patient for treatment is a human. “Patient” and “subject” are used interchangeably herein.

Pharmaceutically acceptable vehicles: The pharmaceutically acceptable carriers (vehicles) useful in this disclosure are conventional. Remington' s Pharmaceutical Sciences, by E. W. Martin, Mack Publishing Co., Easton, Pa., 15th Edition (1975), describes compositions and formulations suitable for pharmaceutical delivery of one or more therapeutic compounds, molecules or agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease.

Therapeutic: A generic term that includes both diagnosis and treatment.

Therapeutic agent: A composition capable of inducing a desired therapeutic or prophylactic effect when properly administered to a subject. “Incubating” includes a sufficient amount of time for an agent to interact with a cell or tissue. “Contacting” includes incubating an agent in solid or in liquid form with a cell or tissue. “Treating” a cell or tissue with an agent includes contacting or incubating the agent with the cell or tissue.

Therapeutically effective amount: A quantity of a specified pharmaceutical or therapeutic agent sufficient to achieve a desired effect in a subject, or in a cell, being treated with the agent. The effective amount of the agent will be dependent on several factors, including, but not limited to the subject or cells being treated, and the manner of administration of the therapeutic composition.

Pharmaceutical Preparations

Pharmaceutical compositions of the present invention comprise an effective amount of a compound(s) or composition(s) disclosed herein, and/or additional agents, dissolved or dispersed in a pharmaceutically acceptable carrier. The phrases “pharmaceutical” or “pharmacologically acceptable” refers to molecular entities and compositions that produce no adverse, allergic or other untoward reaction when administered to an animal, such as, for example, a human. The preparation of an pharmaceutical composition that contains at least one compound or additional active ingredient will be known to those of skill in the art in light of the present disclosure, as exemplified by Remington's Pharmaceutical Sciences, 2003, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 995, pp. 1289-1329, incorporated herein by reference). Except insofar as any conventional carrier is incompatible with the active ingredient, its use in the pharmaceutical compositions is contemplated.

A composition of the present invention, may comprise different types of carriers depending on whether it is to be administered in solid, liquid or aerosol form, and whether it need to be sterile for such routes of administration as injection. The present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 2003, incorporated herein by reference).

Further in accordance with the present invention, composition(s) of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the pharmaceutical composition may include small quantities of pharmacologically acceptable chelators or co-antioxidants. Examples of chelators include ethylenediaminetetraacetic acid (EDTA) and ethylene glycol-bis(beta-aminoethyl ether)-N,N,N′,N′-tetraacetic acid (EGTA). Examples of antioxidants including gallate esters, ascorbate, vitamin E (or other tocopherols), butylated hydroxytoluene, and/or benzoic acid. These chelators and/or co-antioxidants may be used to stabilize a composition of the present invention. In certain embodiments, these chelators and/or antioxidants may stabilize a composition of the present invention, from decomposition by autooxidation.

In further embodiments, the present invention may concern the use of a pharmaceutical lipid vehicle that includes a composition of the present invention, one or more lipids, and an aqueous solvent. As used herein, the term “lipid” will be defined to include any of a broad range of substances that is characteristically insoluble in water and extractable with an organic solvent. This broad class of compounds is known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, a composition of the present invention may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

Alimentary Compositions and Formulations

In certain embodiments of the present invention, a composition herein, and/or additional agents is formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792,451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof.

Parenteral Compositions and Formulations

In further embodiments, a composition of the present invention, may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

Miscellaneous Pharmaceutical Formulations

In other preferred embodiments of the invention, the active compound may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch.” For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725, 871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045.

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### EXAMPLES

### Example 1

### Clinical Trials

Healthy subjects were supplemented with placebo (vehicle) or natural vitamin E tocotrienol (800 mg) daily for 2 months. Platelet function was assessed using optical aggregometry. Tocotrienols completely inhibited platelet function within thirty (30) days of supplementation when arachidonic acid was used as a platelet agonist.

Following withdrawal, functional platelet aggregation did not return for 20 days in subjects supplemented with tocotrienols. Importantly, platelet function recovery in low-dose aspirin (81mg) treated healthy volunteers occurs within 48-72 hours of withdrawal as shown in FIGS. 1.

### Example 2

### Determining the Safety of Daily 400 mg TCT Supplementation in Stroke or TIA Patients

The oral TCT safety in a stroke/TIA population was studied. This initial critical step is designed to compartmentalize risk, and demonstrate safety in a small stroke/TIA cohort (N=100) receiving anti-platelet and statin medication as the current standard of care.

As shown in FIG. 11, stroke/TIA patients were consented and randomized to PBO or TCT supplementation groups by study biostatistician within 6 months of their sentinel stroke event.

Initial visit: At the initial appointment with the research nurse, the participant returned/signed the consent and HIPAA forms. The Research Nurse recorded the subject's blood pressure and took a baseline blood draw (20 ml) to measure complete blood count, blood glucose, HbA1C, hepatic function panel, platelet function, lipid profile, and vitamin E content. Participants were given a 1-month supply of supplements (400 mg PBO or TCT). Gel-capsules containing TCT or vehicle only (PBO) gel capsules will be provided by Carotech Inc (New Jersey). Subjects were educated regarding possible stroke related symptoms and were instructed to seek medical attention if they experienced such symptoms. They were asked to contact study personnel as soon as feasible following a recurrent event. A baseline health questionnaire that included screening for stroke symptoms was given.

Tape Stripping: Tape stripping is a minimally invasive procedure to test for agent delivery and bioavailability. Following FDA-approved guidelines, the tape stripping site (left forearm) was cleaned by the Research Nurse with an alcohol cleansing pad before the tape strips are applied. The tape stripping procedure consists of discarding two sequential tape strips (Cuderm, TX) from the clean site, followed by collection of ten sequential strippings from the same location that are used for evaluation. Sequential removal of 0.5-1 μm layers of the stratum corneum per strip and does not compromise skin barrier function. Vitamin E content of tape strips was determined by HPLC for compliance purposes as reported by the applicant³⁰.

1-month visit: The Research Nurse performed blood pressure measurement and tape stripping after which subjects will be given another 2-month supply of pills. A brief questionnaire will be conducted to specifically address changes in health (bleeding events, bruising, etc.) potentially related to TCT supplementation.

2, 4, 5, 7, 8, 10, 11-month visits: Subjects were telephoned and a health questionnaire was conducted. Responses to the questionnaire that reflect potentially adverse changes in health prompted an additional clinical follow-up and their primary care physician was notified.

3, 6, 9, 12 -month visit: The Research Nurse recorded the subject's blood pressure and take a blood draw (20 ml) for testing platelet function, complete blood count, lipid profile, blood glucose, HbA1C, hepatic function panel, and vitamin E content. The Research Nurse performed tape stripping for compliance after which subjects were given a 3-month supply of pills (except for the final 12-month appointment). As a secondary compliance measure, subjects were asked to return with the prior month's pill packs for counting.

Results. 400 mg TCT was safely tolerated in stroke survivors. There was no significant difference in liver function panel of healthy participants taking 400 mg TCT daily for 3 months as compared to PBO (Table 2).

TABLE 2

| Hepatic Function Panel | baseline | 3 mos | normal range |
|---|---|---|---|
| PBO (400 mg daily)¹ | | | |
| Albumin (g/dL) | 3.9 ± 0.4 | 3.7 ± 0.4 | 3.4-4.8 |
| Bilirubin Direct (mg/dL) | 0.1 | 0.1 | <0.3 |
| Bilirubin Total (mg/dL) | 0.7 ± 0.2 | 0.7 ± 0.1 | <1.5 |
| Alkaline Phosphatase (U/L) | 82.7 ± 5.0 | 74.7 ± 0.6 | 38-126 |
| ALT (U/L) | 23.0 ± 1.7 | 29.0 ± 8.0 | 8-35 |
| AST (U/L) | 24.0 ± 1.0 | 24.7 ± 3.1 | 5-34 |
| Total Protein (g/dL) | 7.0 ± 0.6 | 6.7 ± 0.3 | 6.4-8.3 |
| TCT (400 mg daily)¹ | | | |
| Albumin (g/dL) | 4.1 ± 0.4 | 4.0 ± 0.2 | 3.4-4.8 |
| Bilirubin Direct (mg/dL) | 0.1 | 0.1 | <0.3 |
| Bilirubin Total (mg/dL) | 0.8 ± 0.2 | 0.6 ± 0.1 | <1.5 |
| Alkaline Phosphatase (U/L) | 86.3 ± 12.7 | 76.3 ± 15.0 | 38-126 |
| ALT (U/L) | 23.0 ± 2.6 | 20.3 ± 2.1 | 8-35 |
| AST (U/L) | 28.0 ± 5.6 | 27.0 ± 6.1 | 5-34 |
| Total Protein (g/dL) | 7.6 ± 0.5 | 7.2 ± 0.2 | 6.4-8.3 |

¹TCP supplemented n = 3, TCT supplemented n = 3.

### Example 3

### Characterizing the Antiplatelet Effect of Oral TCT in Stroke or TIA Survivors Treated with Asprin or Clopidogrel

The inventors investigated whether TCT safely enhances the anti-platelet effect of aspirin and clopidogrel, the two most commonly used anti-platelet agents for secondary stroke prevention.

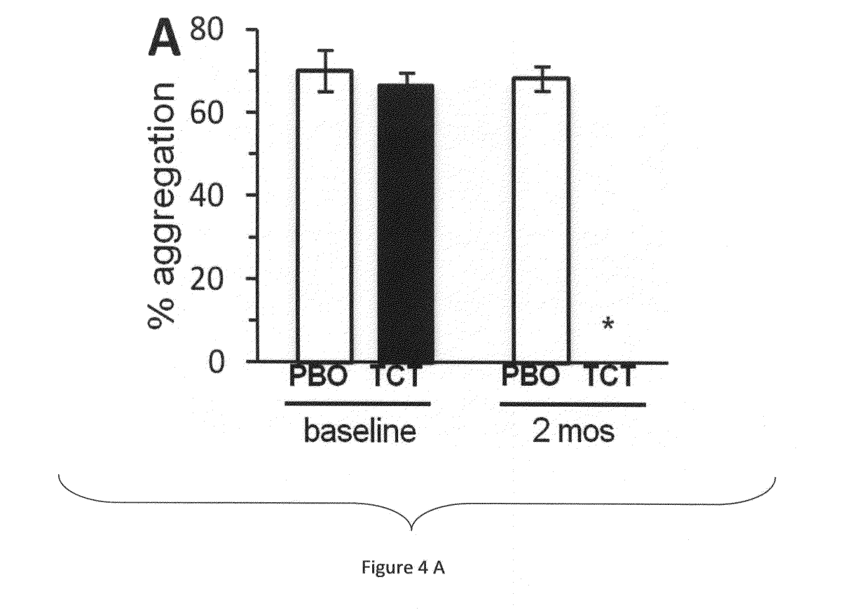

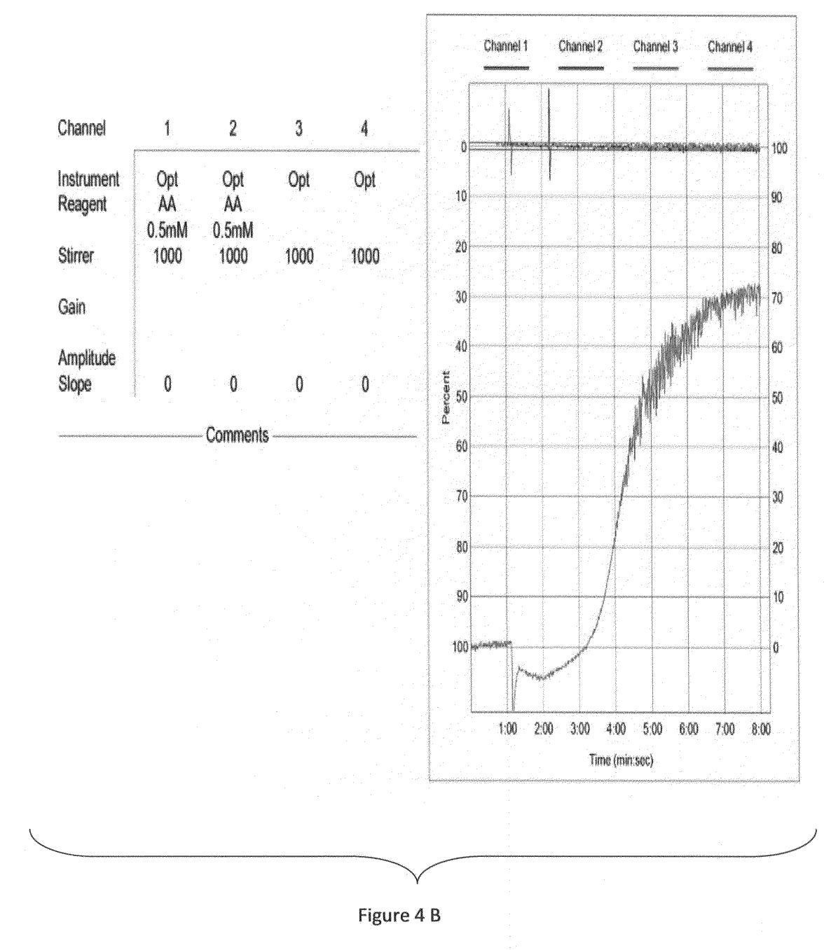

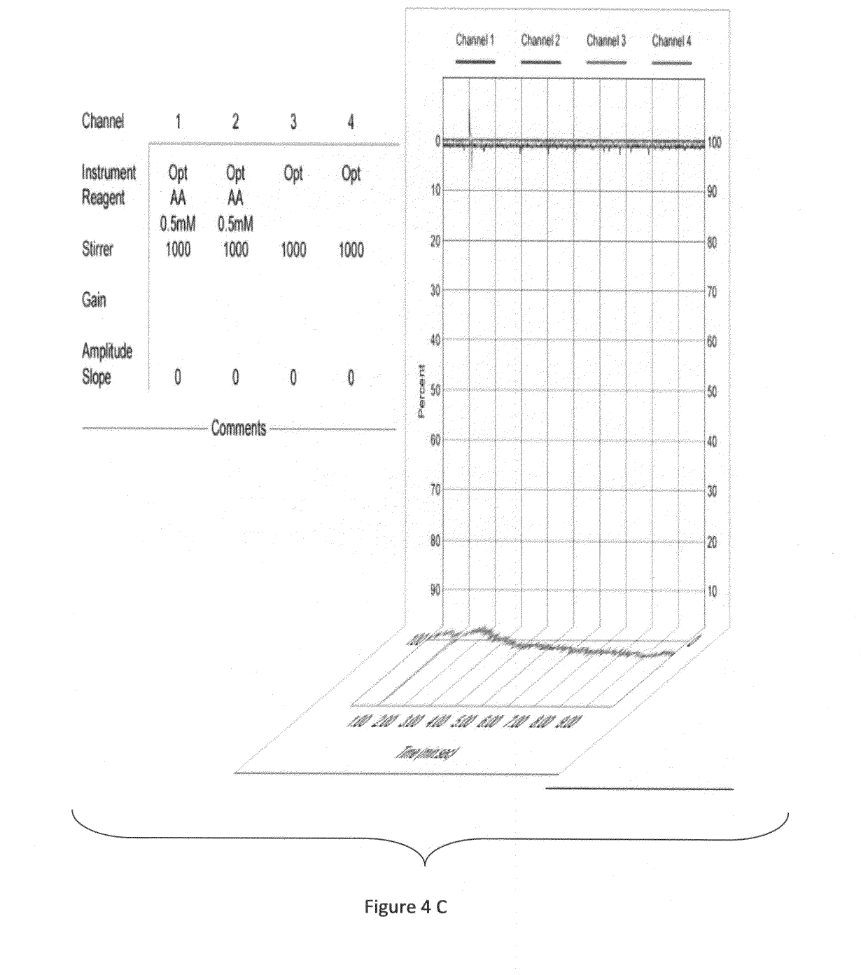

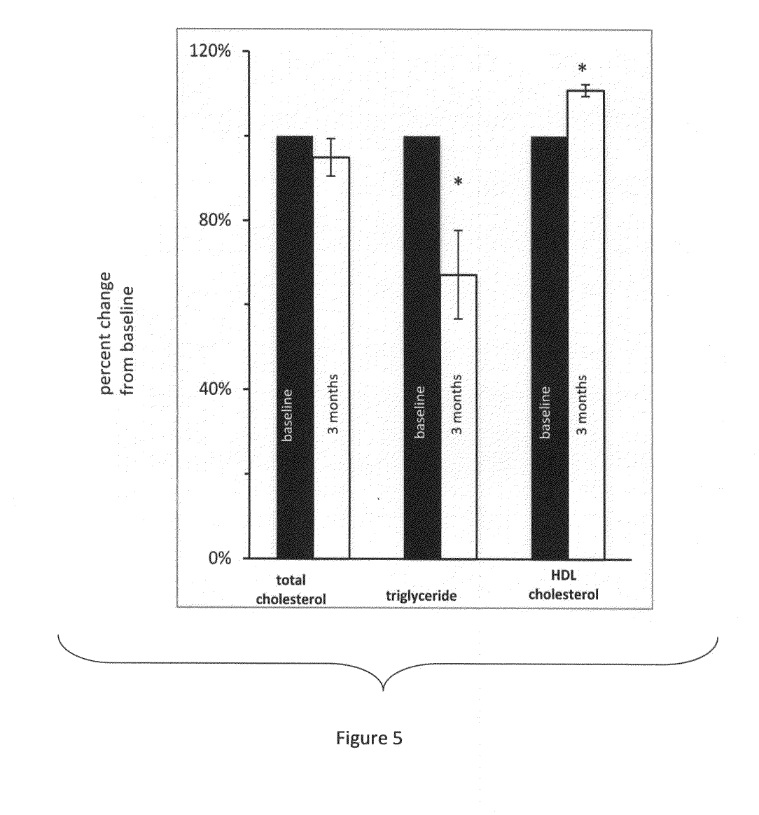

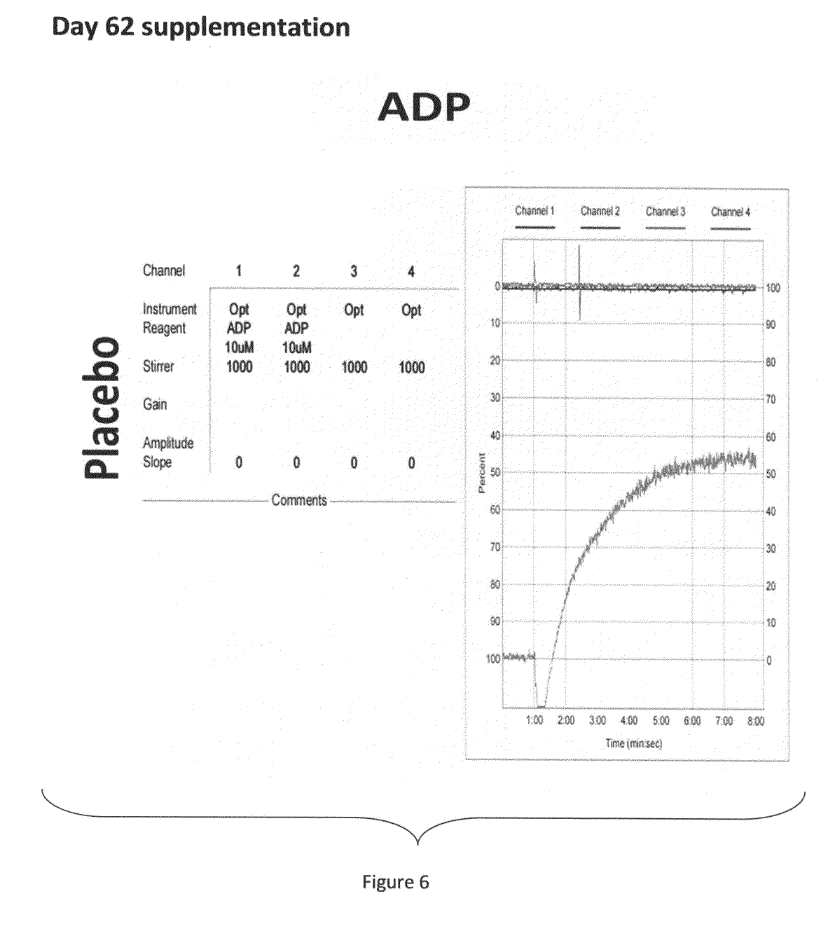

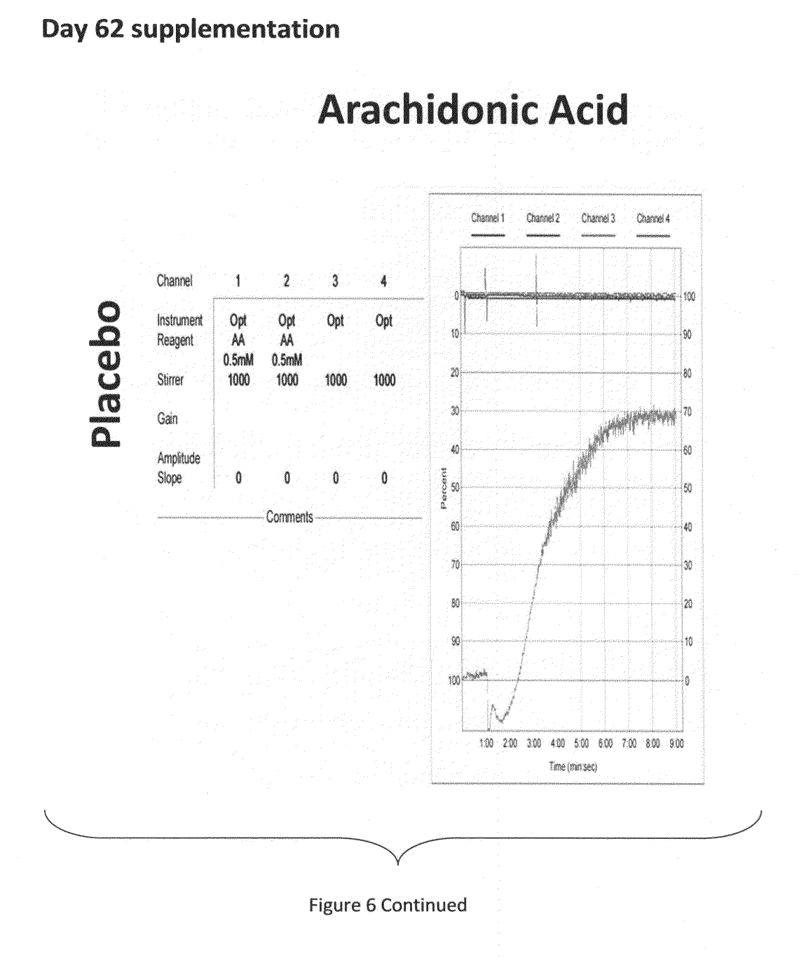

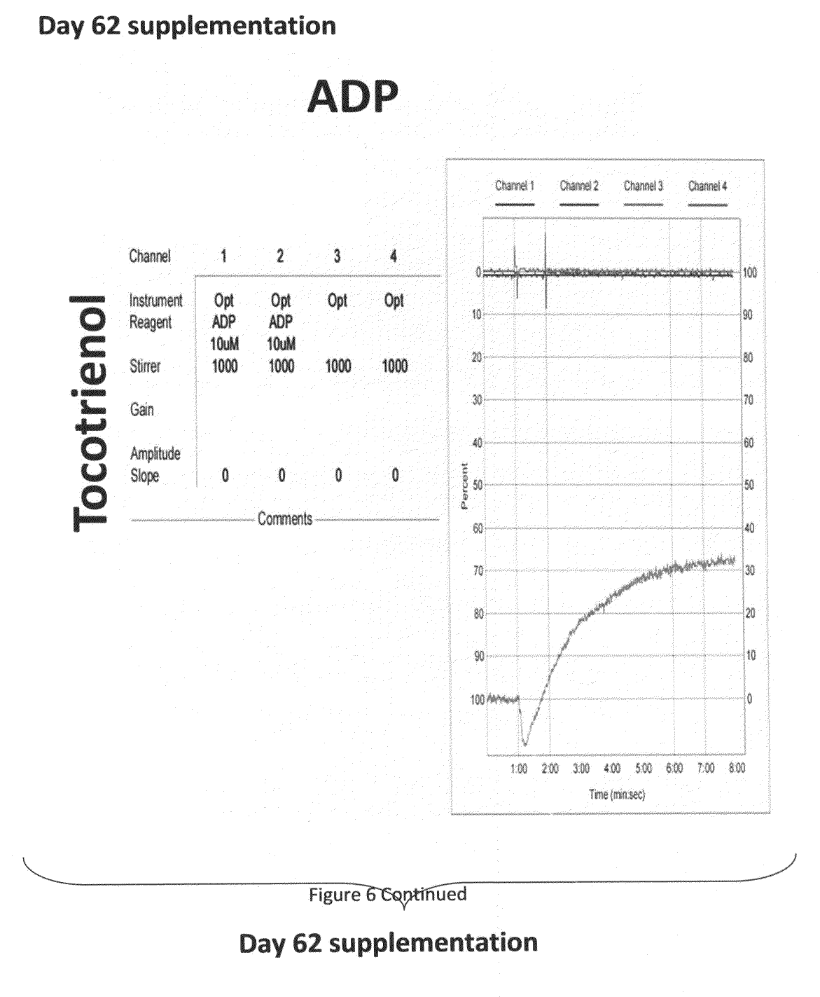

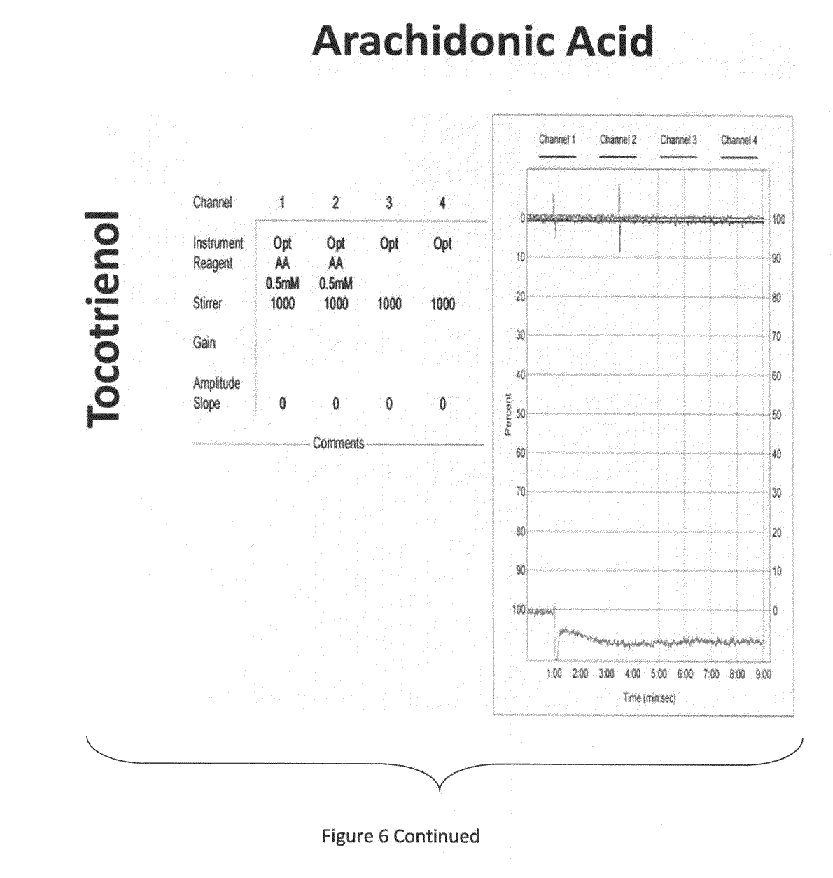

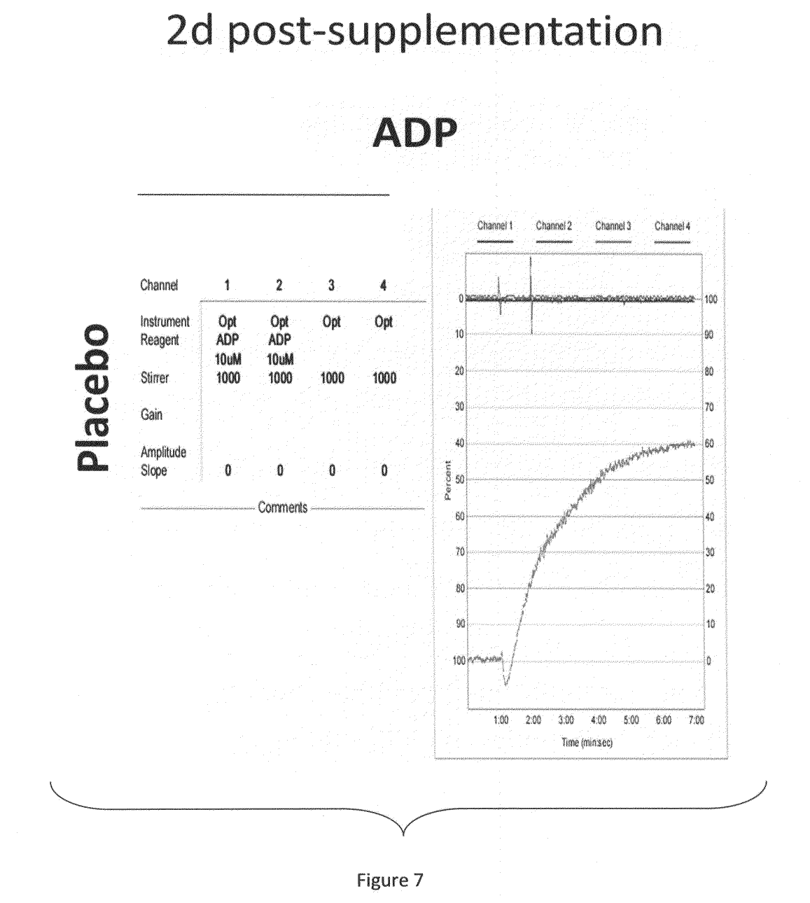

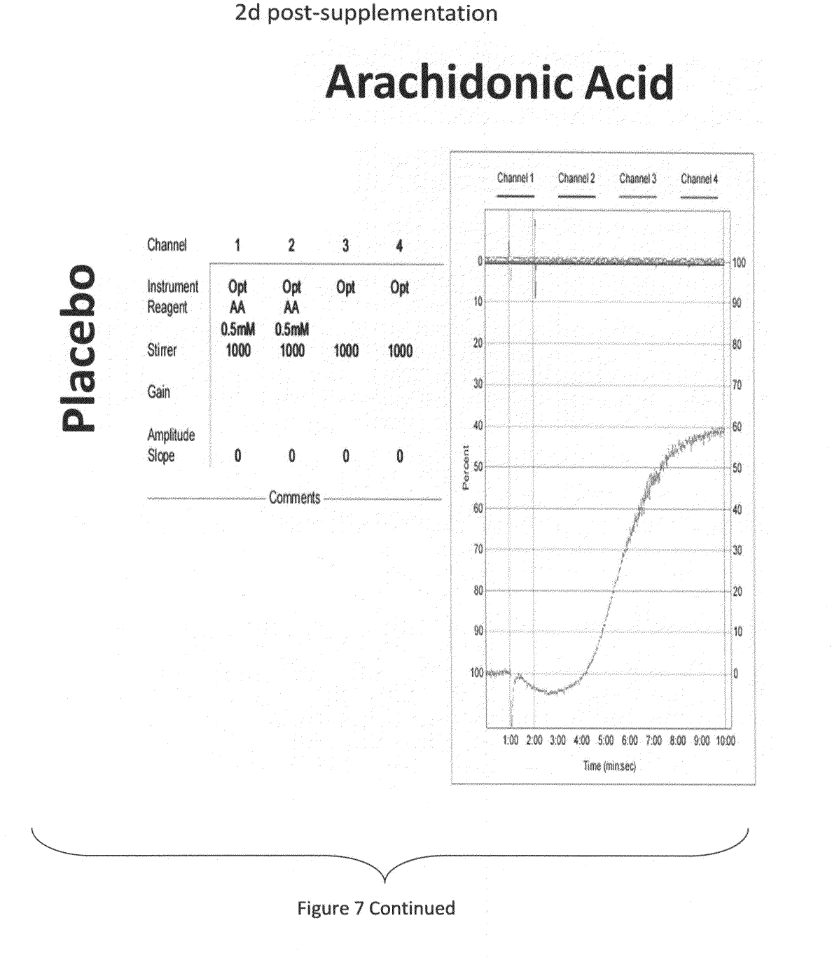

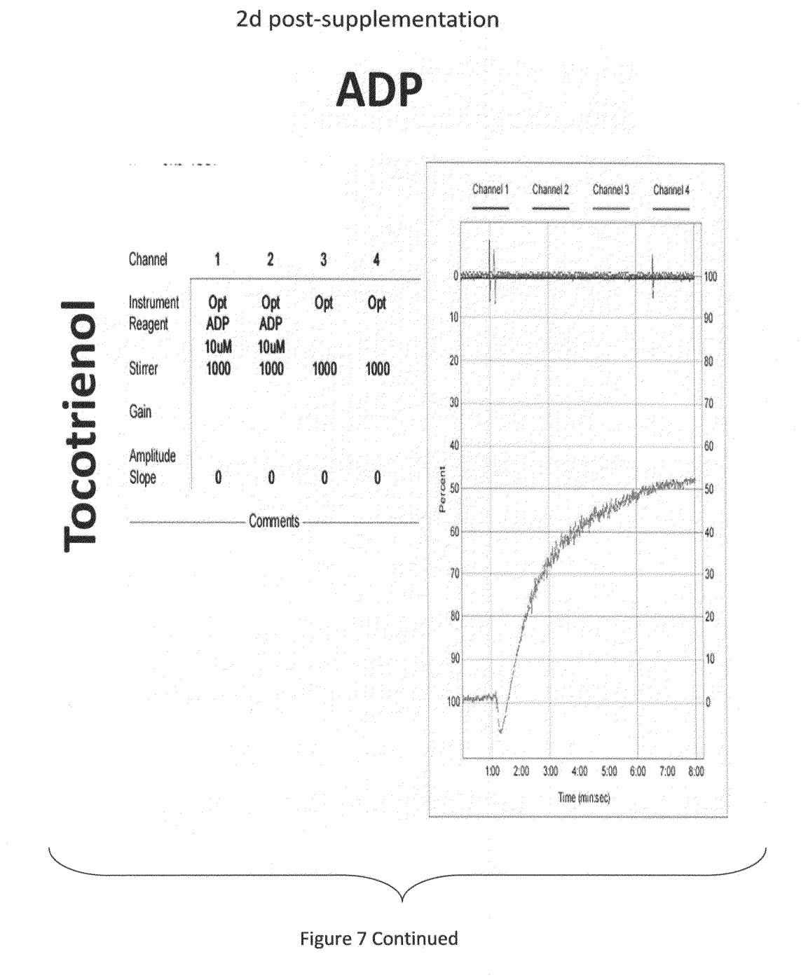

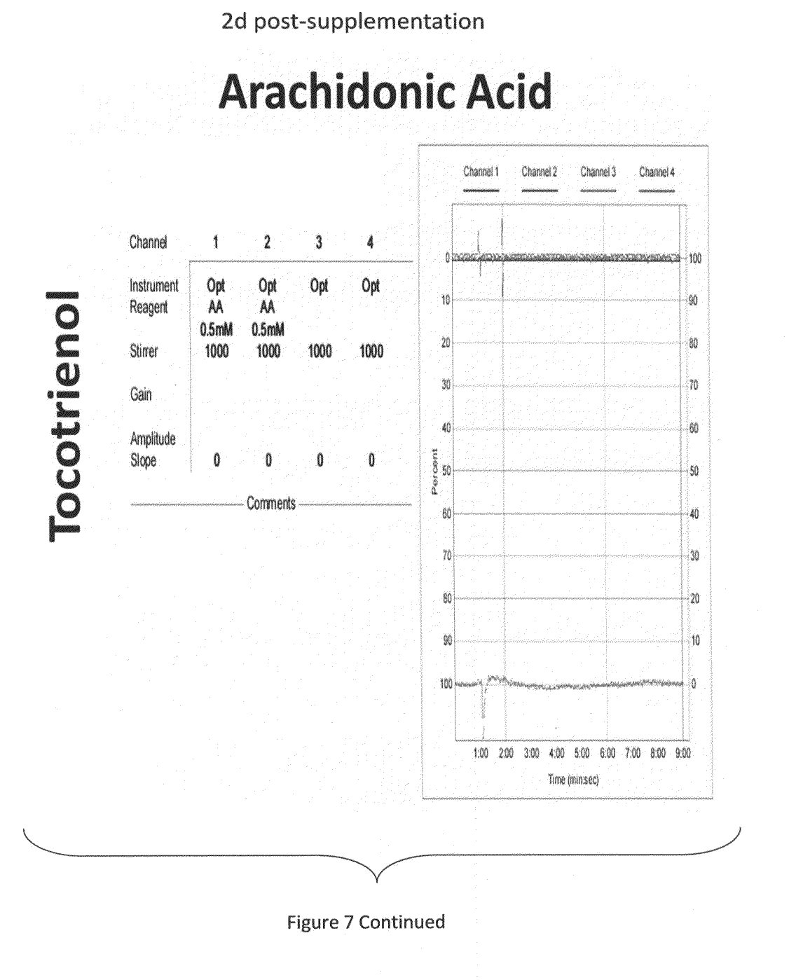

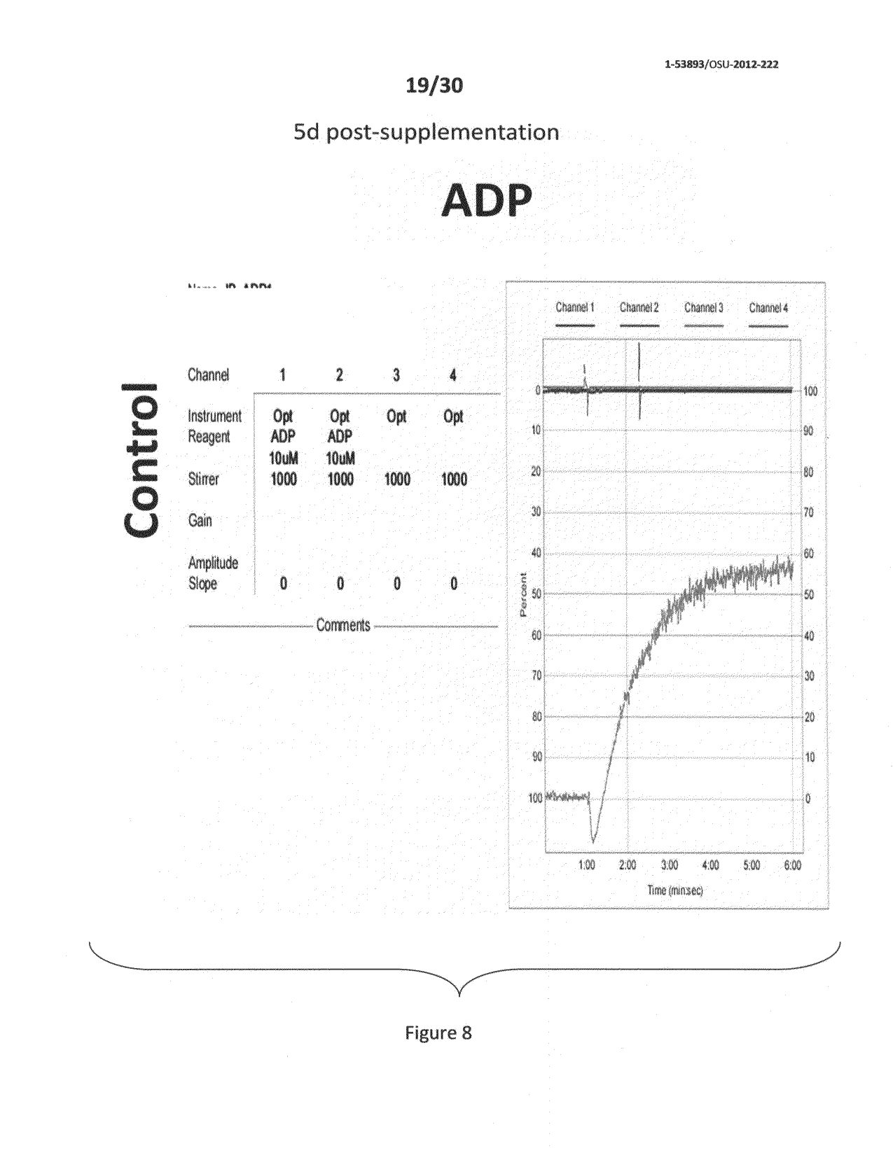

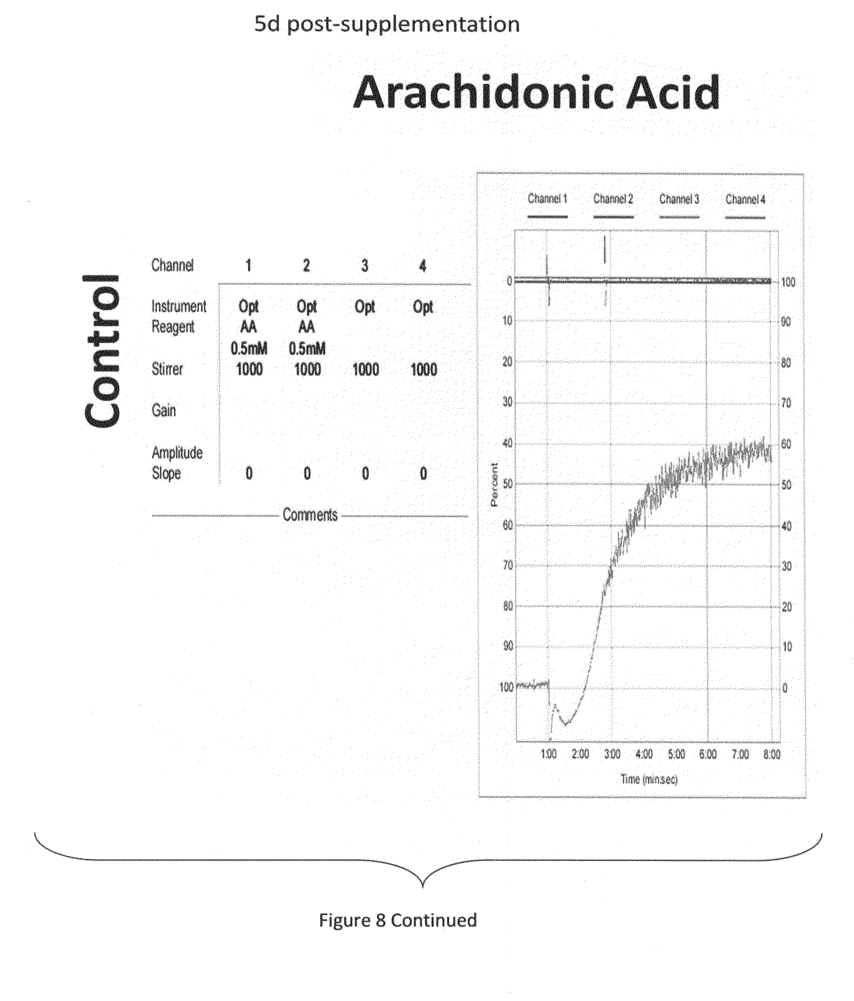

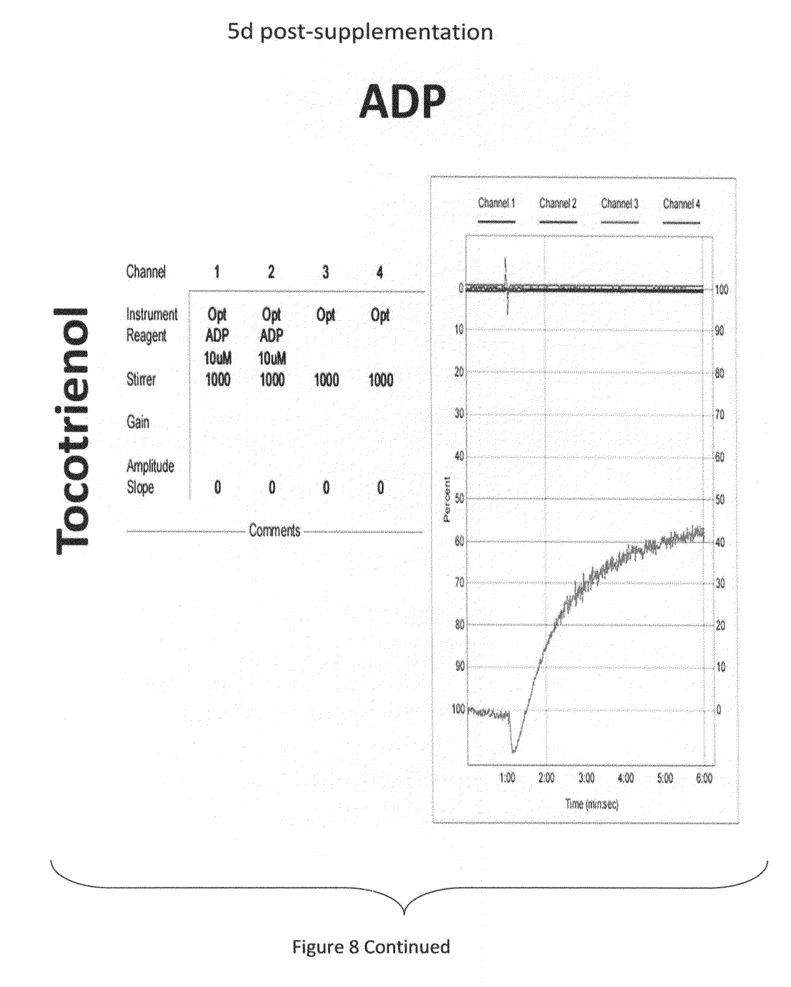

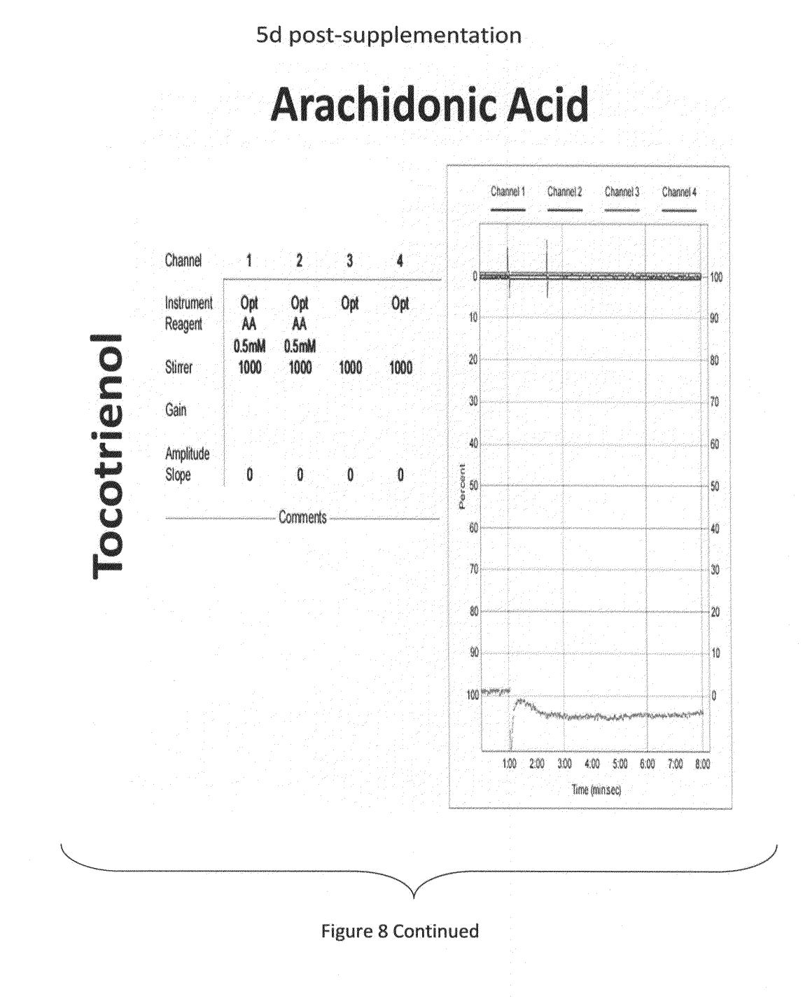

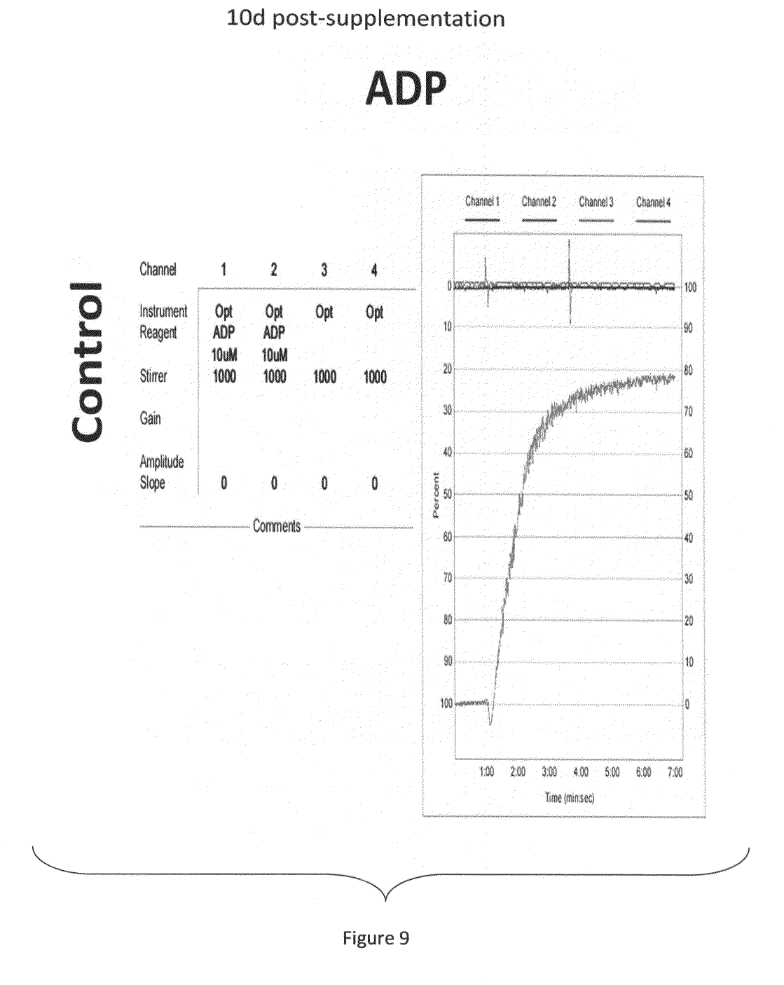

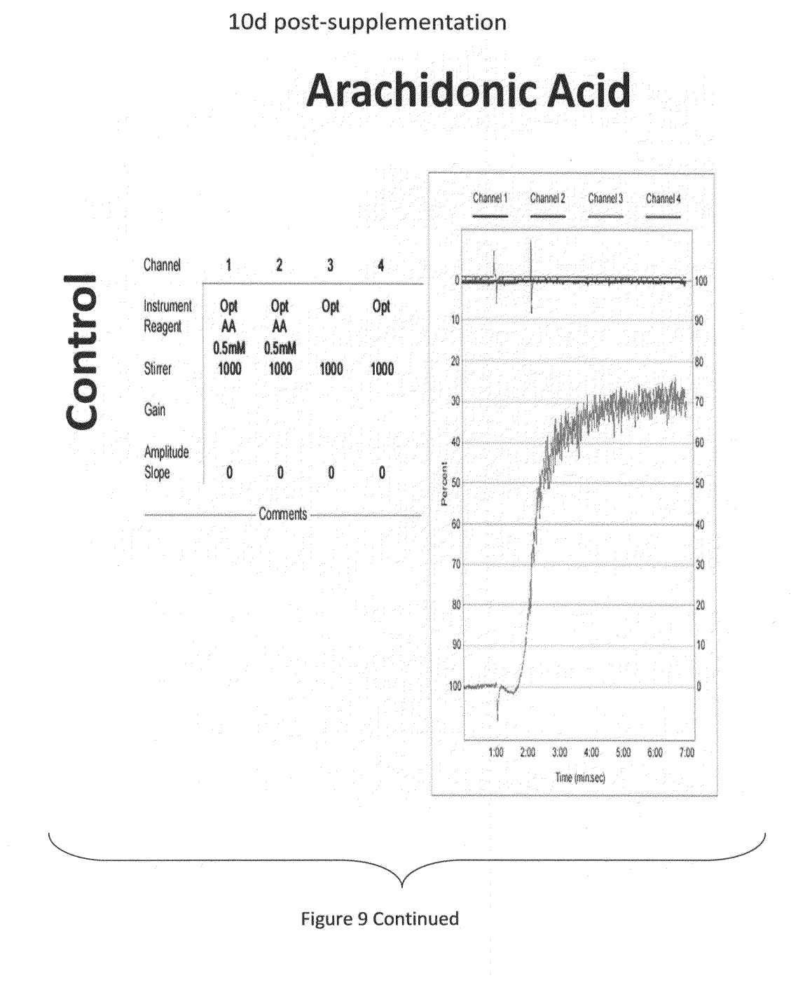

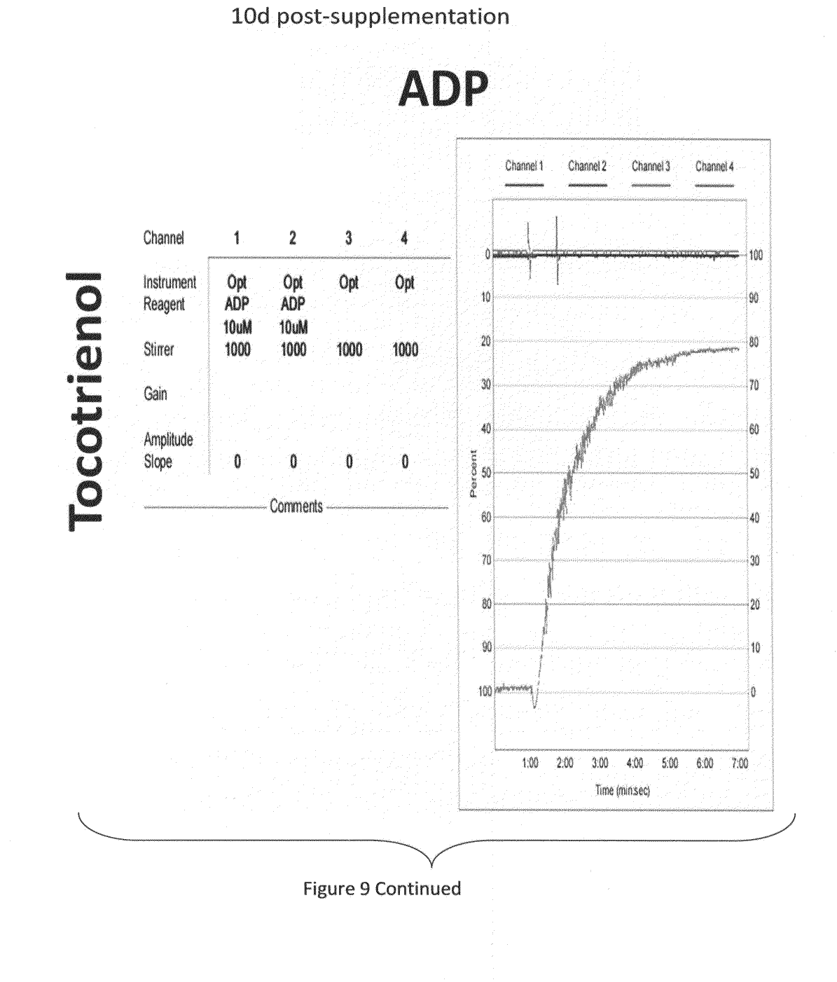

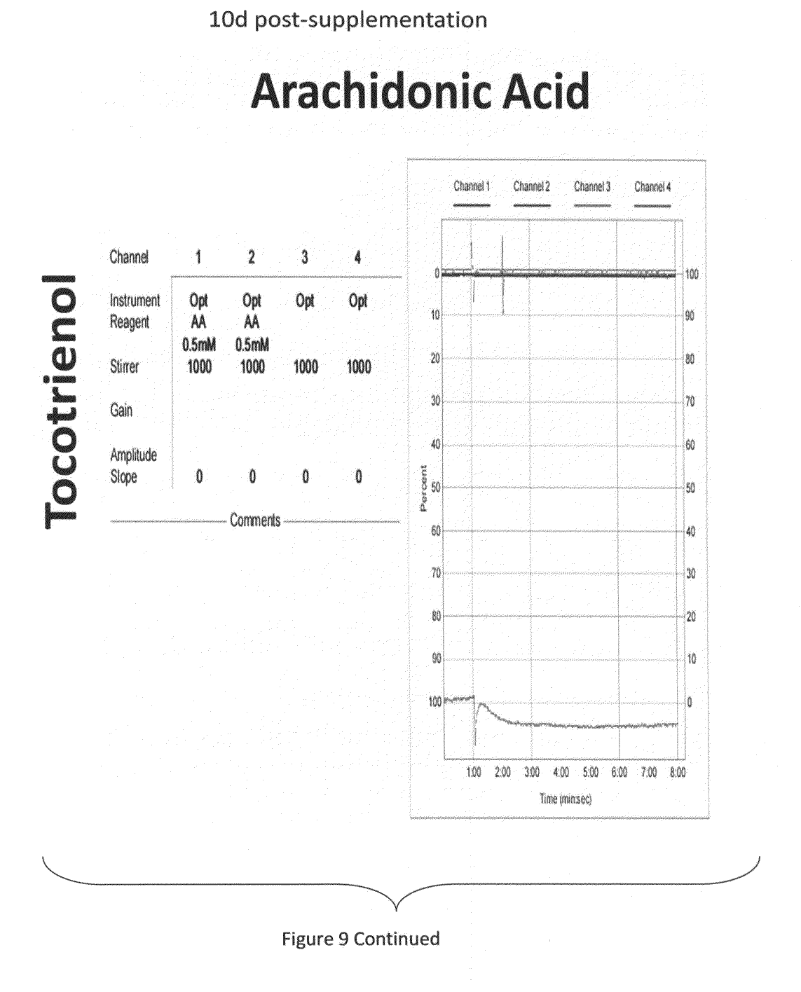

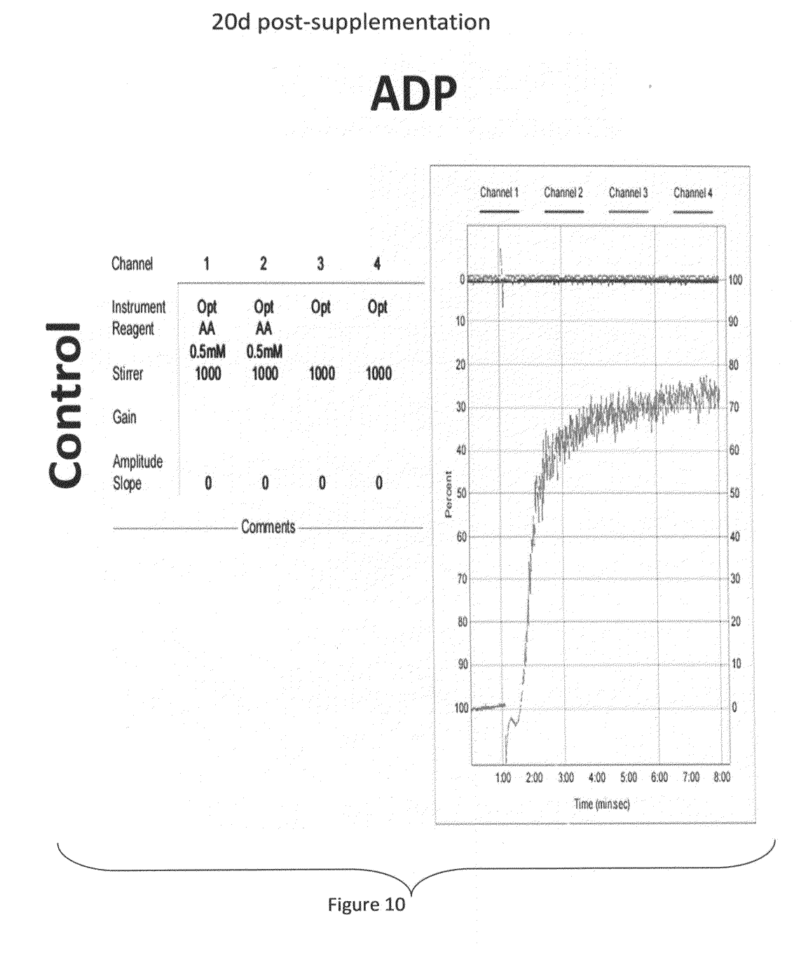

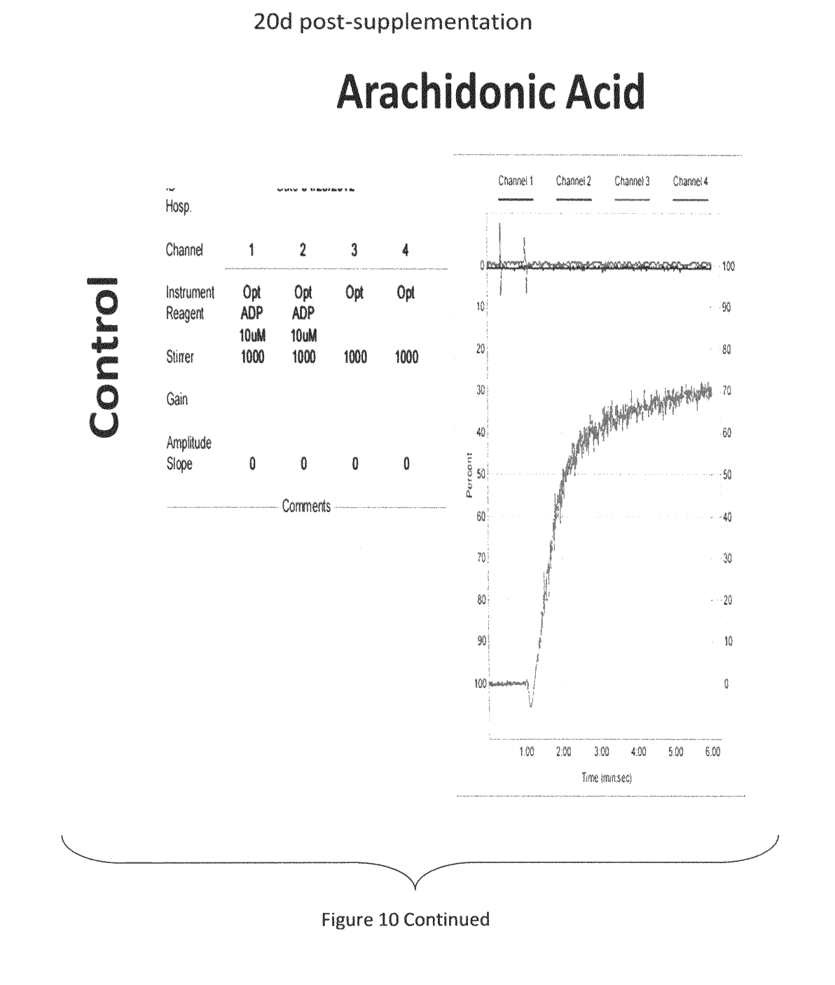

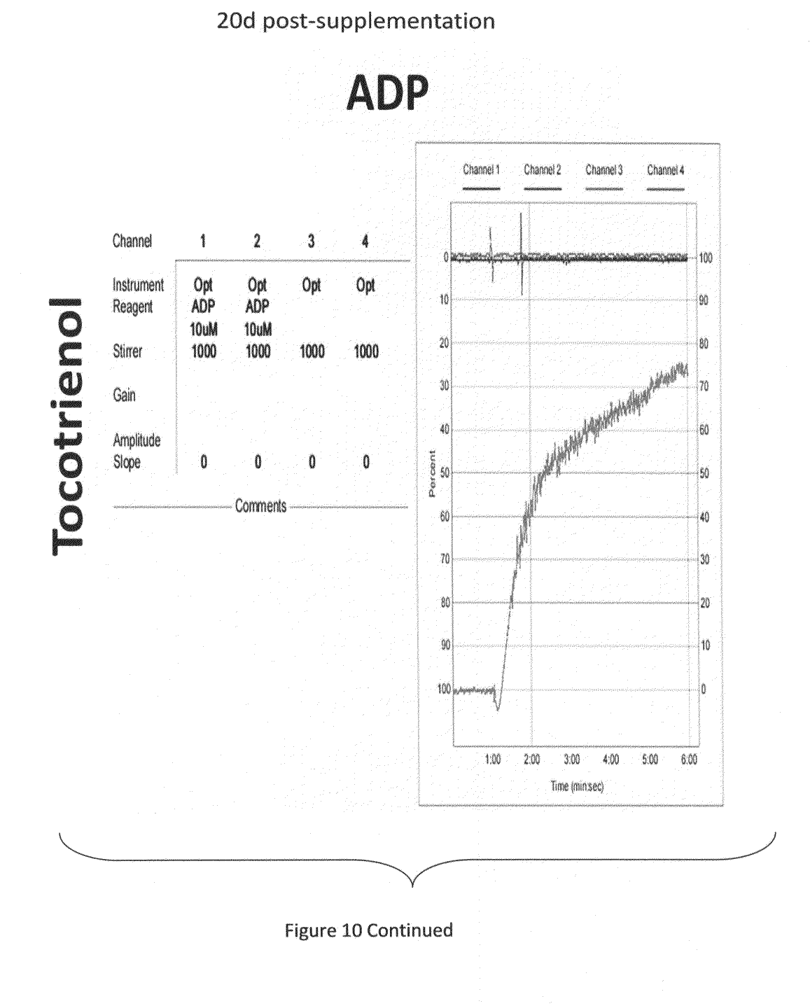

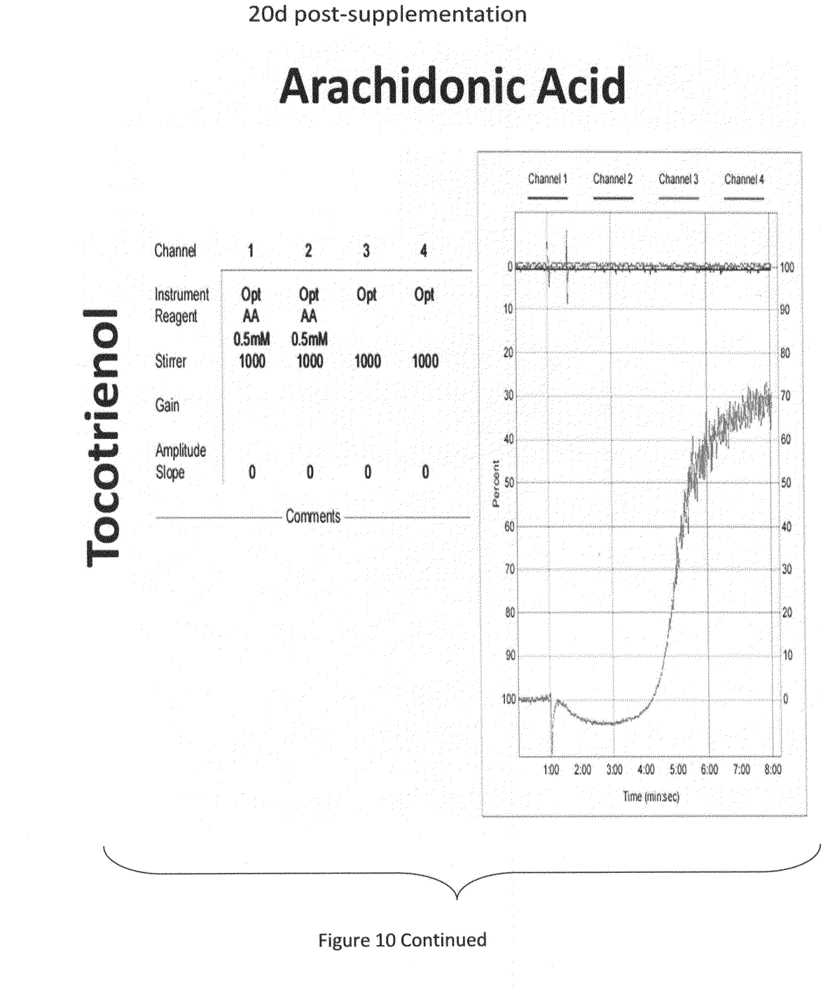

The study design, participant randomization, and sample collection are detailed in FIG. 11. Briefly, whole blood was collected in stroke survivors for aggregometry (FIG. 4) testing at baseline (while on standard of care anti-platelet therapy), and following randomization to PBO or TCT groups at months 1, 3, 6, 9, and 12. Both impedance and optical aggregometry methods were used to assess platelet function. Optical aggregometry from platelet-rich plasma remains the gold standard for platelet function testing, however a growing body of evidence suggests that electrical impedance aggregometry which includes giant platelet subspecies, erythrocytes, and leukocytes more accurately represents in vivo platelet response. Therefore, both optical and whole blood impedance aggregometry was used to assess platelet function using the following agonists: arachidonic acid (0.5 mM), ADP (10 μM), and collagen (2 μg/mL).

To limit premature platelet activation, median cubital venipuncture was performed for whole blood collection using a 20G needle set with a sodium citrate (0.105 M) vacutainer tube (BD Medical, NJ). Blood samples remained at room temperature for 30 min prior to further processing for platelet aggregometry. To limit variability in pH, collection tubes and processing was performed in plasticware that were capped with very little air space relative to blood or isolated platelet volume. Following 30 min, whole blood and isolated platelet optical aggregometry was performed using a Model 700 aggregometer (Chrono-Log Corp., NJ).

Impedance Aggregometry. Whole blood was diluted 1:1 with sterile saline and incubated for 5 min at 37° C. before testing. Electrical impedance aggregometry was recorded under the following settings: 37° C. incubator, 1000 rpm stir bar speed, 0.1× gain. Tracings were recorded for 10 min after the addition of agonists: arachidonic acid (0.5 mM), ADP (10 μM), and collagen (2 μg/mL, Chrono-log Corp., PA).

Optical Aggregometry. After venipuncture (30 min), platelet-rich plasma (PRP) and platelet-poor plasma (PPP) was isolated from whole blood. To obtain PRP, whole blood was spun at room temperature for 15 min at 135 g after which PRP was slowly drawn off using a 1 mL pipette. The remaining sample was spun again for 15 min at 1500 g. PPP was collected and stored separately using a 1 mL pipette. Optical aggregometry measures the increase in light transmission through a stirring suspension of isolated platelets upon addition of agonists. PPP served to establish 100% light transmission for the instrument. PRP at baseline was zeroed to 0% transmission. After the addition of agonists (arachidonic acid, ADP, and collagen as above), tracings were recorded for 10 min.

Results. In healthy subjects, daily oral TCT (400 mg) significantly attenuated arachidonic acid-induced platelet aggregation (FIG. 4). Oral TCT enhanced anti-platelet response as compared to PBO and decrease the incidence of aspirin and clopidogrel resistance without increased bleeding risk. TCT is a natural and safe anti-platelet combination therapy in patients identified at baseline as aspirin and/or clopidogrel non-responders.

### Example 4

### Does TCT Reduce the Incidence of Recurrent Stroke in Survivors

The inventors investigated whether TCT in addition to currently prescribed anti-platelet therapy will further decrease stroke recurrence in stroke/TIA patients.

The study design, participant randomization, and sample collection are outlined in FIG. 11. On the basis of published data, a stroke recurrence rate of 11% after one year was expected.

Patients were instructed to contact study personnel as soon after a recurrent stroke as possible. Hospital records and copies of brain imaging studies were obtained for review and an added follow-up visit scheduled as soon as feasible. Patients were seen and examined by the study team stroke neurologist, Dr. Andrew Slivka who was blinded to treatment status. After the evaluation and review of records and imaging he determined whether the subject had experienced a stroke. In any subject who may not have sought treatment for a mild stroke, the health questionnaire given at all the clinic and phone follow-ups had questions designed for stroke screening. Any positive responses to these questions prompted a clinical evaluation by Dr. Slivka and testing including brain imaging to determine whether a stroke had occurred.

Data analysis. Statistical analyses was performed using SAS 9.3 (SAS Institute, Inc., Cary, N.C.). Descriptive statistics (mean, median, standard deviation, frequency, etc.) for collected variables was determined. The incidence of life threatening and major bleeds as well as the incidence of possible statin related side effects (including elevated liver function greater than 2 times normal, development diabetes, myalgias requiring stopping medication) was compared between the 2 groups using Chi-square testing. For the platelet function analysis, four results obtained through the course of the study were compared with the baseline. In patients who are anti-platelet resistant at baseline, the percent of patients with at least two of four follow-up results revealing platelet aggregation inhibition will be compared between each of the treatment groups and the control group using Chi-square test. Also the percent of patients with at least two of four treatment aggregation tests that demonstrated more than 10% greater inhibition compared to baseline were compared between each of the treatment groups and controls using the Chi-square test. Mean LDL levels were compared between the groups at baseline and average LDL levels during the course of the study were compared between the groups using analysis of variance. The incidence of stroke occurring during the course of the study was compared among the 3 groups using Chi-square testing.

Results. This is the first evidence of a natural anti-platelet and statin mimetic vitamin with the ability to reduce stroke incidence. TCT in addition to current anti-platelet therapies reduces stroke recurrence. Given known benefits of pharmacological anti-platelet and statin therapies in reducing recurrent stroke incidence, TCT stands is a natural anti-platelet and statin mimetic adjunct.

While a number of exemplary aspects and embodiments have been discussed above, those of skill in the art will recognize certain modifications, permutations, additions and sub-combinations therefore. It is therefore intended that the following appended claims hereinafter introduced are interpreted to include all such modifications, permutations, additions and sub-combinations are within their true spirit and scope.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims. Whenever a range is given in the specification, all intermediate ranges and subranges, as well as all individual values included in the ranges given are intended to be included in the disclosure. When a Markush group or other grouping is used herein, all individual members of the group and all combinations and subcombinations possible of the group are intended to be individually included in the disclosure.

In general the terms and phrases used herein have their art-recognized meaning, which can be found by reference to standard texts, journal references and contexts known to those skilled in the art. The above definitions are provided to clarify their specific use in the context of the invention.

All patents and publications mentioned in the specification are indicative of the levels of skill of those skilled in the art to which the invention pertains. Supplementary information is available at the Journal of Cerebral Blood Flow and Metabolism website and said reference by the inventors is hereby incorporated by reference.

What is claimed is:

1. A method of reducing blood clotting in a subject in need thereof, comprising administering a pharmaceutically-effective formulation of at least one isoform of a tocotrienol and reducing blood clotting in a subject.

2. The method of claim 1, wherein the formulation comprises a dose of tocotrienol selected from the group consisting of: approximately 10 mg to approximately 1000 mg per day; approximately 50 mg to approximately 500 mg per day; approximately 100 mg to approximately 500 mg per day; approximately 200 mg to approximately 500 mg per day; approximately 300 mg to approximately 500 mg per day; up to approximately 400 mg per day.

3. The method of claim 1, wherein the formulation comprises a dose of tocotrienol selected from the group consisting of: approximately 200 mg to approximately 1000 mg in one weekly dose; approximately 300 mg to approximately 1000 mg in one weekly dose; approximately 400 mg to approximately 500 mg in one weekly dose.

4. The method of claim 1, wherein the formulation is administered as a dose regimen selected from the group consisting of: approximately every other day; approximately every third day; approximately every fourth day; approximately every fifth day; approximately every sixth day; approximately every seventh day; approximately every other week; approximately once a month; approximately twice a month; approximately three times a month; approximately four times a month; approximately five times a month; approximately six times a month; approximately seven times a month; and approximately eight times a month.

5. The method of claim 1, wherein the formulation is an orally-delivered supplement.

6. The method of claim 3, wherein wherein the oral supplement is delivered by one or more of: a capsule, a tablet pill, a colloid, a piece of chewing gum, a gel, a drink, a food additive, a thin film dissolving strip, an emulsified food spread, an emulsion, a syrup, a meat food, a dairy food, and an egg.

7. The method of claim 1, wherein the subject is selected from a population at elevated risk for a blood clotting pathology selected from the group consisting of: transient ischemic attack (TIA); stroke; embolism; and cardiac arrest.

8. The method of claim 1, wherein the subject is selected from the group consisting of: wherein the subject is selected from: a person who has suffered a previous blood clotting pathology; a person who has suffered a stroke; a person who has suffered a cardiac event; a person who has suffered from transient ischemic attack; a person who has suffered an embolism; a person who has suffered a thrombosis; a person with a genetic predisposition for pathologic blood clotting; a person with biomarkers for pathologic blood clotting; and a person who takes anticoagulants.

9. The method of claim 1, wherein the subject is selected from the group consisting of: wherein the subject is selected from: astronaut, pilot, professional racecar driver, deep-sea diver, mountain climber, pre-surgery patient, sickle-cell anemia patient, sleep apnea patient, drug rehabilitation patient, elderly person, elderly animal, greyhound, racehorse, or companion animal.

10. A pharmaceutical composition for the treatment of blood clotting disorders comprising:
one or more antiplatelet-aggregation drugs, and
one or more tocotrienol compounds.

11. The pharmaceutical composition of claim 8, wherein the thrombolytic drug is selected from the group consisting of: aspirin; clopidogrel; and dipyridamole.

12. A method of reducing reducing risk of a second stroke in a stroke patient in need thereof, comprising administering a pharmaceutically-effective formulation of at least one isoform of a tocotrienol and reducing risk of a second stroke in a stroke patient in need thereof.

13. A method of claim 10, wherein the stroke patient is resistant to aspirin or clopidogrel.

14. A method of claim 10, which comprises administering up to 400 mg of at least one tocotrienol for at least 30 days, and a pharmaceutically-effective dose of at least one tocotrienol weekly thereafter.

15. A method of reducing reducing risk of a second stroke in a stroke patient in need thereof, comprising administering a pharmaceutically-effective formulation of at least one isoform of a tocotrienol and reducing risk of a second stroke in a stroke patient in need thereof.

16. A method of claim 15, which further comprises administering to the patient a pharmaceutically-effective amount of aspirin or clopidogrel.

17. A method of claim 13, which comprises administering up to 400 mg of at least one tocotrienol for at least 30 days, and a pharmaceutically-effective dose of at least one tocotrienol weekly thereafter.

18. A method of inhibiting arachidonic acid-mediated/12-LOX related platelet aggregation in a subject in need thereof, comprising administering a pharmaceutically-effective amount of mixed tocotrienols at weekly.

19. A method for ameliorating the risk or symptoms of pathologic platelet aggregation in a subject in need thereof and for which aspirin is contraindicated, comprising administering a pharmaceutically-effective amount of mixed tocotrienols to the subject.

20. A method of claim 19, wherein aspirin is contraindicated for a reason selected from the group consisting of: alcoholism; liver pathology; clotting disorder; allergy; salicylate intolerance; asthma; bronchospasms; peptic or intestinal ulcers; diabetes; gastritis; hemophilia; glucose-6-phosphate dehydrogenase deficiency; dengue fever; children or adolescents; kidney disease; hyperuricemia; and gout.

21. A method for ameliorating the risk or symptoms of pathologic platelet aggregation in a subject in need thereof and for which clopidogrel is contraindicated, comprising administering a pharmaceutically-effective amount of mixed tocotrienols to the subject.

22. A method of claim 19, wherein aspirin is contraindicated for a reason selected from the group consisting of: alcoholism; liver pathology; allergy; poor metabolizer due to cytochrome p450 CYP2C19 variant; stomach or intestinal ulcer; and kidney disorder.

23. A method for reducing the risk of a heart attack in a subject in need thereof, administering a pharmaceutically-effective amount of mixed tocotrienols at weekly.

24. A method for reducing the risk of a heart attack in a subject in need thereof, administering a pharmaceutically-effective amount of mixed tocotrienols at weekly.

25. A composition as substantially described herein with reference to and as illustrated by the accompanying drawings.

26. A method as substantially described herein with reference to and as illustrated by the accompanying drawings.
